(19) United States
(12) Patent Application Publication
(10) Pub. No.: US 2024/0416337 A1
(43) Pub. Date: Dec. 19, 2024

(54) Quantitation of chemical, biological and biochemical analytes on a flow assay using a smartphone

(71) Applicants: Stuart Farquharson, Meriden, CT (US); Chetan Shende, Ellington, CT (US); Duncan Farquharson, Meriden, CT (US)

(72) Inventors: Stuart Farquharson, Meriden, CT (US); Chetan Shende, Ellington, CT (US); Duncan Farquharson, Meriden, CT (US)

(73) Assignee: Real-Time Analyzers, Inc., Middletown, CT (US)

(21) Appl. No.: 18/742,202

(22) Filed: Jun. 13, 2024

Related U.S. Application Data
(60) Provisional application No. 63/508,615, filed on Jun. 16, 2023.

(51) Int. Cl. B01L 3/00; G01N 33/53; G01N 33/543; G01N 33/569

(52) U.S. Cl. CPC B01L 3/5023; G01N 33/5308; G01N 33/54388; G01N 33/56983; B01L 2200/025; B01L 2200/16; B01L 2300/021; B01L 2300/0609; B01L 2300/0825; B01L 2400/0406; G01N 2333/165; G01N 2469/10; G01N 2470/04

(57) ABSTRACT

The present invention provides a novel method and apparatus that employs a signal-enhancing probe in combination with a flow assay strip to bind a target analyte with substantial selectivity, sensitivity, speed, and convenience, through the use of target analyte-specific binding agents and smartphone camera measurement and analysis. The present invention is advantageous for detecting, identifying, quantifying, and analyzing target analytes, such as chemical, biochemical, or biological substances, in analyte samples. The present invention is most effective in detecting, identifying, quantifying, and analyzing drugs, bacterial and viral pathogens in body fluids, such as blood, blood plasma, exhaled breath, nasopharyngeal mucus, and saliva.

### STATEMENT OF GOVERNMENT INTEREST

The United States Government has rights in this invention under DoD contracts Nos. W81XWH19-C-0079 and W81XWH20-C-0037.

### BACKGROUND OF THE INVENTION

The ability to measure trace quantities of chemical, biochemical, and biological analytes in a variety of matrices is important to numerous fields. Examples range from quantifying the amount of drugs in blood plasma to quantifying the amount of infectious agents in nasopharyngeal samples. In most cases samples are collected and measured by in-house hospital laboratories or independent laboratories. While there are a wide range of drugs used to treat medical conditions, many require continuous concentration monitoring to be safe and effective. Examples include chemotherapy drugs, immunosuppressant drugs, and opioid addiction treatment drugs.

In the case of the approximately 0.6 million USA organ transplant patients, immunosuppressant drugs, such as tacrolimus, prednisone, sirolimus, and azathioprine, are required to keep the immune system from rejecting the transplanted organ. However, the dosage and regiment of these drugs must be monitored to ensure that concentrations are sufficient to avoid rejection, yet not produce toxic effects, such as renal damage and neurotoxicity. This is challenging since the immune system and drug pharmacokinetics are patient dependent. In the case of tacrolimus, also known as FK506, the general protocol is trough drug levels at 10 to 15 ng/ml for the 1^{st} 5 month induction phase, and 5 to 10 ng/ml for the maintenance phase (Kershner R, Fitzsimmons E, “Relationship of FK506 whole blood concentrations and efficacy and toxicity after liver and kidney transplantation”, Transplantation, 62, 920-926, 1996). However, these concentrations have to be monitored every week during the post-operation induction phase to set the patient dependent concentrations, and at least once a month to monitor stability during the maintenance phase for the rest of their lives.

This requires patients to make frequent visits to hospitals or clinics to have their drug-plasma concentrations measured. The analysis involves multiple steps: drawing blood, centrifugation to separate the plasma from the red and white blood cells, and analysis by complex and expensive laboratory instruments, such as liquid chromatography to separate the drug from the plasma, and tandem mass spectrometers to further isolate and quantify the drug (LC-MS/MS, Kalt DA “Tacrolimus: A review of laboratory detection methods and indications for use”, Lab Medicine, 48, e62-e65, 2017). This technology is labor intensive, takes 3-6 hours to perform the measurement, and often a few days for doctors and patients to obtain results.

The combination of potential transplant rejection, patient specific dosing, complex lab measurements, frequent, multi-hour long medical clinic visits, and painful blood draws, places a significant burden and emotional strain on the patient, which has led to nonadherence of drug taking schedules as high as 39%, which in turn is associated with very high transplant failures and death in greater than 90% of patients (Dew M A, et al., “Rates and risk factors for nonadherence to the medical regimen after adult solid organ transplantation”, Transplantation, 83, 858-873, 2007 and Al-Sheyyab A, et al. “Association of medication non-adherence with short-term allograft loss after the treatment of severe acute kidney transplant rejection”, BMC Nephrology, 20, 373, 2019) (Laederach-Hofmann K, Bunzel B, “Noncompliance in Organ Transplant Recipients: A Literature Review”, Gen Hosp Psychiatry, 22, 412-424, 2000).

The quality of transplant patient lives could be substantially improved if a simple, painless, at-home measurement of their immunosuppressant drugs existed. In the case of most treatment drugs the measurement must also be specific and sensitive. Recent studies have shown that tacrolimus concentrations in saliva are similar to blood and can be used to monitor treatment (Hamadi S. et al. “Saliva versus blood therapeutic drug monitoring of tacrolimus in Jordanian kidney transplant patients”, Nov Appro Drug Des Dev 4, 1-5, 2018; Ghareeb M, et al., “Tacrolimus concentration in saliva of kidney transplant recipients: factors influencing the relationship with whole blood concentrations”, Clin Pharmacokinet 57, 1199-1210, 2018). Tacrolimus does not ionize at physiological pH, and readily passes through salivary glands into the oral cavity (Haeckel R, “Factors influencing the saliva/plasma ratio of drugs”, Ann NY Acad Sci 694, 128-142, 1993). This is ideal, since the unionized drug, i.e. the free form, is responsible for the therapeutic effect, as well as toxicity (Zahir H, et al., “Changes in tacrolimus distribution in blood and plasma protein binding following liver transplantation”, Ther Drug Monit, 26, 506-515, 2004). Finally, saliva is safe to handle, sampling is painless and can be performed by a non-expert.

The concept of measuring drugs in saliva is not new, and there exist many commercial lateral and vertical flow assay (LFA and VFA) test kits (T-Cube 6 panel oral fluid drug tests, Transmedeco, accessed Feb. 22, 2022 at: https://transmedco.com/tcube-6-panel-oral-fluid-drug-test/; OralTox, Premier Biotech, accessed Feb. 22, 2022 at: https://premierbiotech.com/innovation/rapid-testing/oral-fluid-testing/oraltox/; 6-panel-oral-saliva-drug-test, Uritox, accessed Feb. 22, 2022 at: https://www.drugtestpanels.com/collections/oral-saliva-drug-testing/products/6-panel-oral-saliva-drug-test). However, these kits are not quantitative, and providing only a visible “test line”, that indicates the presence of a drug, usually by class.

For more than 20 years we have been investigating the ability of surface-enhanced Raman spectroscopy (SERS) to measure drugs in saliva (Farquharson S, et al., “SERS method and apparatus for rapid extraction and analysis of drugs in saliva”, U.S. Pat. No. 7,393,691 B2, 2008, Farquharson S, et al. “Analysis of 5-fluorouracil in saliva using surface-enhanced Raman spectroscopy”, J Raman Spectrosc. 36, 208, 2005; Farquharson S., et al., “Quantitative measurements of codeine and fentanyl on a surface-enhanced Raman-active pad”, Molecules, 24, 2578-2585, 2019; and Farquharson S, et al., “Analysis of drugs in saliva of US military veterans treated for substance use disorders using supported liquid extraction and surface-enhanced Raman spectroscopy”, Molecules, 28, 2010, 2023). SERS employs a laser to generate a plasmon field at the surface of gold or silver nanoparticles, and at the same time amplify the Raman scattering from molecules within that field by as much as 6 orders-of-magnitude (Jeanmaire D L, RP Van Duyne, “Surface Raman Spectroelectrochemistry”, J Electroanal Chem, 84, 1-20, 1977; or Weaver M J, S Farquharson, M A Tadayyoni, “Surface-enhancement factors for Raman scattering at silver electrodes: Role of adsorbate-surface interactions and electrode structure”, J Chem Phys, 82, 4867-4874, 1985).

Recently, we developed a rudimentary LFA that incorporated a SERS-active pad as the test line and measured fentanyl in saliva at 500 ng/ml (Shende C, et al., “Detection of codeine and fentanyl in saliva, blood plasma and whole blood in 5 minutes using a SERS flow-separation strip”, Analyst, 144, 5449-5454, 2019). However, as previously noted, tacrolimus requires quantitation at much lower concentrations from 5 to 15 ng/mL. Consequently, we investigated the ability to further amplify the Raman scattering at the LFA pad by adding a dye molecule to generate surface-enhanced Resonance Raman spectroscopy (SERRS, Farquharson, S and C Shende, “Detection of Tacrolimus in saliva using a lateral flow assay and surface-enhanced resonance Raman scattering”, J Anal Bioanal Tech, 13:3, 1-4, 2022).

During development of the tacrolimus SERRS assay, it was found that the LFA test line showed differences in intensity as a function of concentration that were visible to the naked eye. This was entirely unexpected, as the SERRS probes (henceforth referred to as signal-enhancing probes), consisting of synthesized gold nanoparticles, coated with a dye, as a reporter molecule, and functionalized with antibodies specific to tacrolimus, were designed to amplify Raman scattering by approximately 9-orders-of-magnitude, such that a laser-based Raman spectrometer could detect such low ng/ml concentrations. Follow-up tests showed that a visible spectrometer could be used to measure the intensity of the probes at the test line. Furthermore, a simple, inexpensive apparatus was designed to determine if a series of tacrolimus samples could be measured using an LFA, a smartphone camera and a software application contained on the smartphone (henceforth the smartphone software is referred to as an App). The ability of a smartphone with a camera and an App to quantify target analytes on lateral or vertical flow assay strips (FAS) is the subject matter of this patent application. It is worth noting that the smartphone adds the capability of a patient to perform the measurement at-home and transmit the measured results to their physician, healthcare provider and other recipients, such as a medical database.

The use of a smartphone to detect target analytes on FAS is not new. One of the first applications was the detection of drugs-of-abuse. The smartphone included a holder with an LED to illuminate fluorescent probes on the test line (Carrio A, et al., “Automated low-cost smartphone-based lateral flow saliva test reader for drugs-of-abuse detection, Sensors, 15, 29569-29593, 2015). This combination is used by many smartphone-LFA apps (Nelis J L D, et al., “Smartphone-based optical assays in the food safety field”, Trends in Analytical Chemistry, 129, 115934, 2020), but they only provide a yes/no presence of the target analyte. Since the COVID-19 Pandemic, a number of researchers have developed assays to detect this and other pathogens using a smartphone. For example, the amount of SARS-CoV-2 RNA with a florescent tag was biochemically multiplied using clustered regularly interspaced short palindromic repeats (CRISPER) to the point of detection by a smartphone, and then the initial concentration was back-calculated based on the amount of reagents used (Ning B. et al. “A smartphone-read ultrasensitive and quantitative saliva test for COVID-19”, Science Advances. 7, 2, 2021). However, the amplification required expensive reagents, lab equipment, and took over 2 hours. Other smartphone assays that provide quantitative results include fairly sophisticated optics, to which the smartphone physically connects. This approach has been used for several biomarkers and drugs (Zheng Q. Wu, H, Haiyan J, Yang J, Gao Y, “Development of a smartphone-based fluorescent immunochromatographic assay strip reader”, Sensors, 20, 4521, 2020). None of these current smartphone-read LFAs are capable of both at-home measurements and quantitative analysis, especially single digit ng/mL concentrations.

### SUMMARY OF THE INVENTION

It is the broad object of the present invention to provide a novel method and apparatus for detecting, identifying, quantifying and analyzing target analytes, i.e., chemicals, biochemicals, or biological substances in analyte samples, using a lateral or vertical flow assay strip (FAS) and a smartphone at the point-of-need, point-of-care, and especially at-home.

It is a more specific object of the invention to provide such a method and apparatus wherein detection, identification, quantitation and analysis are effected by measuring the intensity obtained from, but not limited to one of the following; (a) the test line, and (b) the test line and the control line of a FAS using a smartphone camera, wherein the FAS employs probes that provide superior signal enhancement and are functionalized with analyte-specific binding agents, resulting in substantial sensitivity, selectivity, speed and convenience. Examples of signal-enhancing probes include nanoscale gold or silver particles or colloids, coated with dye or thiol molecules, encapsulated in silica or a polymer, and functionalized with binding agents. Examples of probe binding agents include antibodies, peptides, enzymes, nucleic acid sequences, or antigens, designed to bind to the corresponding target analytes, such as a drug or pathogen, a structural or molecular subunit of the drug or pathogen, a protein or nucleic acid sequence, or antibodies raised to inactivate a pathogen.

It has now been found that the foregoing and related objects of the invention are attained by the provision of a method and apparatus in which a target analyte is bound between two analyte specific binding agents to form a sandwich structure, wherein the first analyte specific binding agent, or agents, is attached to the surface of a signal-enhancing probe, and the second analyte specific binding agent is attached to the surface of a FAS. The FAS will in most cases consist of a sample pad where the sample is added, a conjugate pad containing the probes with the first analyte specific binding agent, a test line containing the second analyte specific binding agent, a control line containing a probe specific binding agent, and a wicking pad to which the sample flows. The probe and test line binding agents are of the same type, and may be the same, while the control line binding agent is of the opposite type.

In practice an analyte sample in liquid form is added to the sample pad of a FAS, wherein the sample flows, driven by capillary action, first across the conjugate pad containing the probes, which become mobile, mix and bind to the target analytes, if present, second, the probe-target analyte conjugates flow across the test line functionalized with the second analyte-specific binding agent, wherein the conjugates bind if they have bound target analytes forming a sandwich structure, and third, the sample continues to flow towards the wicking pad, passing across the control line functionalized with a probe-specific binding agent, wherein unconjugated probes bind. In practice, the FAS is most often a lateral flow assay strip (LFAS) incorporated into a plastic or cardboard cassette used in the horizontal position, and sometimes the FAS is a vertical flow assay strip (VFAS) used in the vertical position, such as dipping the strip into a liquid sample. In practice the intensity of the FAS test line and control line are measured using a smartphone camera that provides the following information: (a) a measurable intensity at the control line indicates selective binding of the probes, and that the FAS performed properly, (b) no measurable intensity at the control line indicates that the FAS failed to perform properly, (c) an unmeasurable intensity at the test line and a measurable intensity at the control line indicates no or undetectable binding of the target analyte. (d) measurable intensities at the test and control line indicate selective binding of the target analyte at test line and selective binding of the probes at the control line, which in turn indicates positive detection and identification of the target analyte. (e) comparison of the measured intensities at the test line alone or the test line and the control line to a corresponding calibration curve provides quantitation of the target analyte.

In practice developing a calibration curve to quantify said target analyte is performed by using smartphone measurements of a series of analyte samples of known concentration encompassing the quantitative range of the target analyte based on, but not limited to one of the following: (a) the intensity of the test line, (b) the intensity of the test line divided by the intensity of the control line, and (c) the intensity of the test line divided by the intensity of the test line plus the intensity of the control line.

More specifically, certain objects of the invention are attained by the provision of a method for detection, identification, quantitation and analysis, using a FAS and a smartphone, of “at least one” designated target analyte, comprising the steps: providing a probe composed of a metal, coated with a reporter molecule, encapsulated in a silica outer layer, that is functionalized with “at least one” a first binding agent that has a specific capability for binding thereto “at least one” a designated target analyte; providing a FAS comprised of a material supporting, in sequence, a sample pad for sample deposition, a conjugate pad containing the probes, a test line functionalized with a second binding agent that also binds the target analyte, a control line functionalized with a third, different type of binding agent to bind excess probes, and a wicking pad, all of which have sufficient porosity to promote flow by capillary action from the sample pad to the wicking pad; obtaining an analyte sample, possibly containing said “at least one” designated target analyte; adding said analyte sample to the FAS; establishing or maintaining conditions sufficient to effect sample flow and binding of said “at least one” designated target analyte to said “at least one” first binding agent of said functionalized probes at the conjugate pad; establishing or maintaining conditions effective to cause the target analyte now bound to a probe to flow and bind to a second binding agent at a test line forming a sandwich structure; establishing or maintaining conditions effective to cause excess functionalized probes to flow and bind to a third binding agent at the control line; providing a smartphone containing a camera capable of displaying and recording a real-time image of the flow assay strip; providing a smartphone App to measure the intensity obtained from, but not limited to the recorded image of one of the following: (a) the test line, and (b) the test line and the control line; quantifying said target analyte by comparing the smartphone measured intensity obtained from, but not limited to the recorded image of one of the following: (a) the test line, and (b) the test line and the control line to a corresponding concentration calibration curve contained in the App; said corresponding concentration calibration curve predeveloped from smartphone measurement of intensities of a series of analyte samples of known concentration encompassing the quantitative range of the target analyte, based on, but not limited to one of the following: (a) the intensity of the test line, (b) the intensity of the test line divided by the intensity of the control line, and (c) the intensity of the test line divided by the intensity of the test line plus the intensity of the control line; wherein, the intensity is one of, but not limited to the following measured at the test line and the control line: (a) the absorbance, (b) the absorption, (c) the optical density, (d) reflectance, (e) the reflection, and (f) the red, green, and blue color values individually and in any combination.

In preferred embodiments, analysis is obtained by using the test line and the control line as follows: a measurable intensity at both lines indicates positive detection and identification of the target analyte, a measurable intensity at only the control line indicates negative detection of the target analyte. and no measurable intensity at the control line indicates a failed measurement.

In some preferred embodiments, the calibration curve to be used to quantify a target analyte, is downloaded to the smartphone by scanning a barcode or a QR code on the sample package using the aforementioned App on the smartphone.

In preferred embodiments, the quantified target analyte is then used to calculate the concentration of the target analyte in appropriate terms, including, but not limited to ng/ml, ng/cc, mg/L, part per billion (ppb), part per million (ppm), colony forming units (cfu), cycle-to-threshold (Ct) values, and the like.

In some preferred embodiments, the calculated concentration is expressed in general terms, such as low, high, good, bad, not-infected, infected, not contagious, contagious, and the like.

In preferred embodiments, the analysis is displayed on the smartphone screen in an easy to understand format, and preferably can be exported manually or automatically using the smartphone to text, email, or phone one or more selected persons and one or more selected databases, directly using the App. Specific examples include (a) sending a treatment drug concentration to the person's physician and their medical provider's database, and (b), the Ct value for a virus, such as SARS-CoV-2 to a person's employer and to a local health agency. Selected persons include one or more friends, families, and co-workers. Selected databases may include one or more hospitals, drug study databases, and health agencies. The latter may include, local, town, city, district, state, regional, and national health agencies.

For some preferred embodiments, the said probes are added to the analyte sample, allowed to bind, and which are then added to a FAS that does not have a conjugate pad containing said probes. Alternatively, the LFAS may be dipped into the sample to which the probes have been added. In either case, the detection, identification, quantitation and analysis are performed as described above.

The probe metal employed in the method of the invention will usually be selected from the group consisting of copper, gold, silver, nickel, platinum, rhodium, iron, ruthenium, cobalt, nickel, palladium, and alloys and mixtures thereof, preferably gold or silver. The metal will normally be of particulate or colloidal form having submicron dimensions.

The reporter molecule employed in the method of the invention will usually have a spectral wavelength absorption in the visible part of the spectrum.

In some preferred cases, the reporter molecule will be a dye or thiol group containing molecule.

In preferred embodiments the said FAS is a substantially planar strip fabricated with a composition selected from the group of nitrocellulose, paper, plastic, a combination of these materials, or any suitable material with sufficient porosity to allow flow.

In some preferred cases the first binding agent will be attached to the silica outer layer of the probe, and the second and third binding agents will be attached to the FAS by means of covalent, ionic, or hydrogen bonding, or by van der Waals or electrostatic interactions between charged, polar, hydrophobic, or hydrophilic chemical groups on the surface of said three binding agents.

In some preferred cases the designated target analyte is attached to said “at least one” first and second binding agent by a means selected from the group consisting of covalent, ionic, and hydrogen bonding, by van der Waals and electrostatic interactions between charged, polar, hydrophobic, and hydrophilic chemical groups on the surface of said “at least one” binding agents.

In some preferred cases the “at least one” first binding agent will be attached to said probe and said “at least one” second and third binding agents will be attached to the FAS by means of covalent, ionic, or hydrogen bonding, or by van der Waals or electrostatic interactions between charged, polar, hydrophobic, or hydrophilic chemical groups on the surface of said three binding agents.

In some cases a linker chemical biochemical or biological is interposed for attaching said “at least one” first binding agent to said probe, and said “at least one” second and third binding agents to said FAS.

In some preferred cases the linker chemical or biochemical will be selected from the group consisting of cysteine, a thiol group of a chemical or biochemical, 4-(N-Maleimidomethyl)cyclohexane-1-carboxylic acid 3-sulfo-N-hydroxysuccinimide ester sodium salt, N-5-azido-2-nitrobenzoyloxysuccinimide, 3-aminopropyl trimethoxy silane, NHS, 1-ethyl-3-(3-dimethylaminopropyl) carbodiimide hydrochloride, and N-hydroxysulfosuccinimide.

In preferred embodiments the said target analyte sample will be obtained from, but not limited to one of the following sources, air, water, soil, humans, animals, food, feed and surfaces.

In some preferred cases the said human analyte sample is selected from the group of exhaled breath, saliva, nasal mucus, throat sputum, blood, blood plasma, blood serum, tears, sweat, urine, feces, or semen, or other biological matter.

In preferred embodiments the said “at least one” designated target analyte and said “at least one” first, second and third binding agents will be chemical, biochemical, or biological substances. The case when said the target analyte is a chemical substance, it is selected from the group consisting of any general chemical, drug, explosive, radionuclide, pesticide, inorganic or organic pollutant, and their associated precursors or break-down products (e.g. hydrolysis products, metabolites, etc.).

In some specific cases the said “at least one” drug is selected from the group consisting of antiepileptic, antiarrhythmic, antibiotic, antidepressants, antidiabetics, bronchodilator, chemotherapy, immunosuppressant, HIV and substance abuse treatment drugs.

In some more specific cases the said “at least one” treatment drug is selected from the group consisting of, but are not limited to: carbamazepine, phenobarbital, phenytoin, valproic acid, digitoxin, digoxin, lidocaine, nacetylprocainamide, procainamide, gentamicin, tobramycin, vancomycin, lithium, theophylline, cyclosporine, mycophenolic acid, azacitidine, 5-fluorouracil, 6-mercaptopurine, capecitabine, cladribine, chlorfarabine, cytarabine, decitabibe, sirolimus, tacrolimus, buprenorphine, methadone, naloxone and naltrexone.

In some preferred cases the said “at least one” target analyte and said “at least one” first, second and third binding agents are a biochemical substance selected from the group consisting of amino and nucleic acids, nucleotides, oligonucleotides, nucleosides, peptides, proteins, lipids, polysaccharides, haptens, antibodies, antigens, affirmer proteins, bacteriophages, biomarkers, enzymes, steroids, hormones, lectins, aptamers, and fragments and polymers thereof (e.g. antibody-fragment, polypeptides).

In some preferred cases the said “at least one” first and second binding agents and said “at least one” designated target analyte are paired with one another for effective inter-bonding, such pairs being selected from the group consisting of (a) antibodies and antigens, (b) peptides and biologicals, (c) drug receptors and drugs, (d) enzymes and their specific biochemical substrates, (e) carbohydrates and lectins, and (f) nucleic acid sequences and their complements.

In some embodiments of the present method the “at least one” target analyte will be a disease-causing, foodborne or waterborne pathogen, or a bioagent. Disease-causing pathogens referred to herein include, but are not limited to Acute Flaccid Myelitis (AFM), Burkholderia mallei, coronaviruses, including Severe Acute Respiratory Syndrome (SARS) -229E, -CoV-2, -HKU1, -OC43, and -NL63, Corynebacterium diphtheriae, Enteric viruses, Enterobacter aerogenes, Equine encephalitis, hemorrhagic fevers, Hepatitus A through E, herpes simplex viruses 1 and 2, human immunodeficiency virus, influenza, Legionella, Lyme borreliosis, measles, meningitis, mumps, methicillin-resistant Staphylococcus aureus (MRSA), Middle East Respiratory Syndrome (MERS), Mycobacterium tuberculosis, Mycoplasma pneumonia, Multisystem Inflammatory Syndrome (MIS), Neisseria gonorrhoeae, Neisseria meningitidis, Norwalk virus, members of the Orthomyxoviridae family, pertussis, Pneumococcal Disease, rabies virus, Respiratory Syncytial Virus Infection (RSV), rhinoviruses, Rubella virus, saxitoxin, sepsis, Shigella subspecies, and dysenteriae, Staphylococcus aureus, Staphylococcal and Streptococcus pneumonia, Swine disease, Treponema pallidum, Vibrio cholerae, Varicella zoster virus, West Nile virus, and Yellow fever.

Foodborne or waterborne pathogens referred to herein include, but are not limited to Aeromonas hydrophilia, Bacillus cereus, Campylobacter jejuni, Clostridium botulinum B and C, Clostridium difficile and perfringens, Cryptosporidium parvum, Escherichia coli, Giardia lamblia, Hepatitis A, influenza, Listeria monocytogenes and its subspecies, Salmonella enterica, typhimurium and its subspecies, Shigella, Yersinia enterocolitica.

Bioagents referred to herein include, but are not limited to Bacillus anthraces, Clostridium botulinum A, Dengue fever, Ebola virus, Francisella tularensis, Leishmania genus, Marburg virus, Mycobacterium leprae, Plasmodium genus, Puumala hantavirus, Ricin toxin, Variola virus, and Yersinia pestis.

In some more specific cases the said “at least one” target analyte is selected from the group consisting of the SARS CoV-2 virion, the spike glycoprotein or the S1 and S2 subunits of the spike protein on the surface of the SARS CoV-2 virion, a membrane protein, the nucleocapsid protein contained inside the envelope of the SARS CoV-2 virion, the RNA and RNA unique sequences of the SARS CoV-2 virus, the antibody created by the human immune system to deactivate the SARS CoV-2 virions, or any subunit or molecular feature unique to the SARS CoV-2 virion.

In certain embodiments of the method establishing conditions to effect binding of said “at least one” designated target analyte to said “at least one” binding agent of said one probe will include pretreatment of the analyte sample prior to addition to the FAS. In some cases pretreatment will include, but will not be not limited to, the use of chemicals, biologicals, filters, heat or light to separate the target analyte from the analyte sample.

Pretreatment chemicals and biologicals are selected from the group consisting of acids, bases, buffers, solvents, digesting agents, lysing agents, coagulants, enzymes, amino and nucleic acids, nucleotides, nucleosides, proteins, lipids, polysaccharides, haptens, antibodies, antigens, affirmer proteins, bacteriophages, biomarkers, steroids, hormones, lectins, aptamers, and fragments and polymers thereof.

Needless to say, sample collection, pretreatment, addition of the sample to a FAS, measuring the intensity at the LFAS's test line, control line, or both of the real-time or recorded image by a smartphone camera, to detect, identify, quantify, and analyze the target analyte, will all desirably be performed with substantial selectivity, sensitivity, and speed. Those desiderata are enabled by the present invention, as described herein.

Additional objects of the invention are attained by the provision of apparatus, in the form of a kit of supplies, for use in the detection, identification, quantitation and analysis of “at least one” designated target analyte in an analyte sample, by a smartphone camera and App, comprising: a packaging means for the containment of a multiplicity of components; “at least one” sample collection device selected from the group consisting of a non-cotton swab, a spatula, a balloon for exhaled breath, an open-ended tube, such as a straw, a vial, a vial containing probes, a centrifuge tube, a cup, a lancet for finger needle prick collection of blood, and a syringe with needle; “at least one” sample holding device selected from the group consisting of a vial, a centrifuge tube, and a cup; “at least one” sample transfer device selected from the group consisting of an eyedropper, pipette, auto pipette, a syringe, a vial, a centrifuge tube, and a cup; “at least one” FAS consisting of a sample pad, with or without a conjugate pad containing probes, a test line functionalized to bind the target analyte, a control line functionalized to bind excess probes, and a wicking pad.

With further regard to the kit of components embodying the apparatus of the invention, such components will usually include, in general terms, means for obtaining an analyte sample; means for treating the sample to effect mutual separation of “at least one” target analyte and interfering chemicals, biochemicals, or biologicals, to produce an analyte sample; means for introducing the analyte sample to a FAS; means to provide sample flow by capillary action; means to provide probes that bind to the analyte sample; means to bind the formed probe-target analytes at a test line to form a sandwich structure; means to bind unbound probes at a control line; optionally, means for introducing a chase buffer to remove any unbound chemicals, biochemicals or biologicals; a means to align a FAS in front of a smartphone camera; a software application means contained on the smartphone (an App); an App means to provide step-by-step instructions for measuring the FAS, including a timer to aid in the performance of the steps; an App means to download the calibration curve by scanning a barcode or QR code on the sample kit, an App means to measure the intensity of the probe reporter molecule at the FAS's test line and the control line using a real-time or recorded image; an App means for calculating the target analyte concentration in the analyte sample using the measured intensity and downloaded calibration curve; and an App means to categorize, display and share the person's determined health based on the determined concentration.

In some preferred embodiments, the “at least one” reagent will be probes that are added to the analyte sample to effect binding prior to addition to a FAS devoid of a conjugate pad containing probes.

In some preferred embodiments said components will also include: “at least one” reagent holding device with a cap selected from the group consisting of a vial, a tube, and a centrifuge tube; “at least one” reagent in a reagent holding device used to pretreat the analyte sample prior to addition to the LFAS.

In some preferred cases the “at least one” reagent is used to separate the target analyte from the analyte sample, wherein the said reagent is selected from the group consisting of acids, bases, buffers, digesting agents, solvents, amino and nucleic acids, coagulants, nucleotides, nucleosides, proteins, lipids, polysaccharides, haptens, antibodies, antigens, affirmer proteins, bacteriophages, biomarkers, enzymes, steroids, hormones, lectins, aptamers, and fragments and polymers thereof.

In some preferred instances the kit will include a chase buffer selected from the group of, but are not limited to water, pH buffers, phosphate buffered saline, NaCl, EDTA, borate, Tween 20, poly(vinyl) alcohol, poly(vinyl) pyrrolidone, surfactants BSA, casein, and other chemicals and biologicals used by those skilled in the art.

In some preferred instances the kit will include a device to separate the target analyte from the analyte sample or from a pretreated sample, selected from the group of a filter, a solid phase extractor, a heater, and a centrifuge.

In some preferred embodiments, the instant apparatus may include a holder, selected from the group of an attachment, a box and a stand that positions the FAS at the smartphone camera measurement point. In some preferred embodiments, the smartphone magnifies and focuses the test line image and as required the control line image for alignment and measurement. An aligning object, selected from the group of a bull's eye, cross hairs, circle, square, rectangle, or the like, is superimposed on the center of the camera view, and is used to define the camera measurement point at which the intensity of the test line and control line are measured by the smartphone App and used to determine the target analyte concertation.

In some preferred embodiments the holder includes a sliding tray to which a cassette holding an FAS is placed, wherein placement of the cassette in the tray in one direction aligns the test line with the camera measurement point, and placement of the tray in the opposite direction aligns the control line with the camera measurement point. This is conveniently accomplished by adding a base to the cassette that is centered on the test and control lines, so that upon insertion into the tray in opposite directions the test line and control line are automatically aligned with the camera.

In some preferred embodiments, the slide automatically moves the cassette holding the FAS from the test line to the control line through communication with the smartphone App.

In some preferred embodiments, the smartphone scans across the test line and control line to detect the maximum intensity of each line, which is used to calculate the target analyte concentration.

In some preferred embodiments, an automated XY positioning stage contains a support with slots that hold multiple FASs, and is used to position the test and control lines for each FAS in front of the smartphone camera measurement point.

In some preferred embodiments the holder may employ a means to block ambient light so that the light on the smartphone camera automatically turns on when the intensity of the test line, control line, or both are measured. In some cases the App on the smartphone, designed for the measurements described herein, controls the intensity of the cameral light.

Ideally, the test kit, holder, and smartphone afford ease of use and portability, for at-site measurements, such as at-home, in an ambulance, at the bedside of a hospitalized patient, at local medical clinics, at the site of an infectious outbreak, at laboratories, and at food processing facilities.

Ideally, the sample flow from the sample pad to the wicking pad occurs within 30 minutes, preferably within 20 minutes, and ideally within 10 minutes.

The chase buffer addition step, if required, will usually require a period of no more than 5 minutes, preferably no more than 1 minute.

The total measurement time of both the test and control lines combined occurs within 1 minute, preferably within 30 seconds, and ideally, within 10 seconds, performed after the sufficient flow of the sample to the wicking pad.

### Definitions

As used in the present application, the following terms and references are to be understood to have the meanings hereinafter set forth, unless a different or further definition is provided, or the context makes it clear that another meaning is intended:

“Chemical substance” means any general chemical, including solvents, drugs, explosives, radionuclides, pesticides, inorganic or organic pollutants, and their associated precursors or break-down products (e.g. hydrolysis products, metabolites, etc.).

“Biochemical substance” means any biochemical involved in chemical processes of living organisms, including amino and nucleic acids, nucleotides, nucleosides, peptides, proteins, lipids, polysaccharides, haptans, antibodies, antigens, affirmer proteins, bacteriophages, biomarkers, enzymes, steroids, hormones, lectins, aptamers, phages, prions, immunoglobulins, toxins, including fragments or polymers thereof (e.g. antibody-fragment, polypeptides). Specific examples include serum albumin, immunoglobulin G, human thyroid stimulating hormone, and prostate specific antigen.

“Biological substance” means a microbial life form, such as any algae, bacteria, fungi, protozoa, or virus.

“Biological matter” means whole blood, blood plasma, blood serum, exhaled breath condensate, breast milk, nasal mucus, nasopharyngeal mucus, saliva, throat sputum, semen, spinal fluid, sweat, tear drops, urine and the like.

As used in respect to a target analyte: “detect” means to obtain a signal from the reporter molecule at the test line, control line, or both; “attach” is exclusively used when describing the composition of the probe. For example, the dye is attached to the metal particle, the binding agent is attached to the silica shell;

“Bind” is exclusively used when describing the binding of a target analyte to a probe, a test line or a control line.

“Smartphone”, herein means a mobile phone with a camera and display screen, with an operating system capable of making phone calls, sending texts, storing data, and accessing the Cloud to upload and download data and use Apps.

“App” herein means a software application that resides on a smartphone and is downloaded from the “Cloud”.

“Cloud” herein means an electronic storage device that contains data, files or software that is publicly or privately accessible through a computer network, including the internet.

“Identify” herein means to determine the chemical, biochemical, or biological identity of the target analyte from selective binding of the signal-enhancing probe and reporter molecule signal generation at the test line.

“Signal” herein means a measureable intensity from the reporter molecule of the signal-enhancing probes.

“Specificity” is used to indicate the selective binding of a particular target analyte, as opposed to analytes with similar structure that would produce a false positive response. Examples of specificity include: (1) selective binding of tacrolimus, but not other drugs, and especially not sirolimus, (2) selective binding of SARS CoV-2, but not other viruses, especially not influenza viruses and most especially not SARS-229E; and (3) selective binding of L. monocytogenes, but not other bacteria, and most especially not other listeria.

Exemplary of the sensitivity that is achieved using the method and apparatus of the invention is the ability to detect and measure 5 ng/ml tacrolimus in saliva, and 106 SARS CoV-2 spike proteins in saliva, equivalent to a polymerase chain reaction (PCR) analyzer cycles-to-threshold (Ct) value of 26, and less than 10 CFUs/cm² L. monocytogenes on a surface.

The analyte sample (i.e., a sample containing a target analyte) utilized in carrying out the method of the invention may be obtained from a broad variety of gaseous, liquid, and solid sources.

Gas sample sources include, but are not limited to air, a gaseous chemical, a chemical or biological aerosol, exhaust fumes, ventilation system or room air, exhaled or ventilated breath, extracted lung air, and mixtures thereof; examples of target analytes in specific gas analyte samples include toxic industrial chemicals in air, and viruses or disease biomarkers in exhaled breath.

Liquid sample sources include, but are not limited to water, chemicals, and biological matter, such as whole blood, blood plasma, blood serum, exhaled breath condensate, breast milk, nasal mucus, nasopharyngeal mucus, saliva, throat sputum, semen, spinal fluid, sweat, tear drops, and urine. Examples of target analytes in liquid analyte samples include a drug in a chemical solvent; bacteria or pesticides in a drink, such as juice or milk; bacteria in a lake, sewer, or water-treatment sample; a pollutant in a lake, river, ocean, ground water, or rain sample; a drug in blood plasma, saliva, or sweat; a virus in exhaled breath condensate, nasal mucus, saliva, or throat sputum; an antibody in blood serum, and a disease biomarker in semen or urine.

Examples of solid sample sources include, but are not limited to food, soil, an animal part, feces, a frozen material, and a substance on a surface. Examples of target analytes in solid analyte samples include bacteria and pesticides in or on fruits, meats, and vegetables; pesticides in soil; poisons in animal kidneys and livers; bacteria in feces; bacterial spores on a mail-sorting machine; explosive materials on or in an improvised explosive device; and drugs and explosives on clothing, luggage, hair, fingertips or fingernails.

The volume of the analyte sample employed will be defined by the required analysis, and can be quite large or very small. For example, cubic meters of air would normally be collected for the purpose of detecting aerosolized bacterial spores, whereas a drop of saliva would normally suffice for the detection of a drug. The volume of the analyte sample employed will generally be quite small, such as 1 to 10 mL; often, however, the volume will be much less.

For many analyte samples the method will desirably include the pretreatment of a collected or sampled material or substance so as to separate the “at least one” target analyte from other components. The residual “other” components will normally constitute all chemicals, biochemicals, and biologicals present in the collected or sampled material that may interfere significantly with analysis. Such interferences include hindering flow of the target analyte(s) to the binding agents, deactivating the probes, and/or producing a signal that would substantially prevent the signal of the “at least one” target analyte from being observed. Such a pretreatment step may include the use of an extracting or degrading chemical or biological substance effective to make the “at least one” target analyte available, and means for separating the “at least one” target analyte from the chemical or biological substance used for extracting or degrading.

In the case of solid analyte samples, the extracting or degrading chemical or biological substance may be selected from the group consisting of acids, bases, buffers, solvents, digesting and lysing agents, and combinations thereof. Examples include the use of a surfactant to break open the envelope of the SARS CoV-2 virion to release the nucleocapsid protein as the target analyte, since it is unique to this coronavirus.

In the case of body samples, the extracting or degrading chemical or biological substance may be selected from the group consisting of solvents, acids, bases, buffers, mucolytic agents, surfactants, and mixtures thereof. Suitable mucolytic agents include N-acetyl-L-cysteine (NALC), Amboxol, Bromhexine, and combinations thereof. For example, a simple acid, like acetic acid can be used to break apart saliva mucans releasing SARS CoV-2 virions for analysis, or a solution of NALC and NaOH is presently preferred for separating drugs, like cocaine, from nasal mucus.

In some cases a combination of reagents and physical separation may be used. For example, the addition of a coagulant to aid the centrifugal removal of red blood cells from blood in the analysis of antibodies in blood serum.

Target analytes, and other components, may be mutually separated from the produced degrading chemical or biological substance using a chemical, physical, or biological method.

Chemical treatment of a sample may employ a solvent for the “at least one” target analyte, which solvent will desirably be of such polarity as to render it capable of extracting the target analyte. Suitable solvents include water containing appropriate acids and bases for pH adjustment; organic liquids such as acetone, acetonitrile, benzene, chloroform, carbon tetrachloride, cyclohexane, dichloromethane, diethyl ether, dimethylsulfoxide, ethyl acetate, ethylene glycol, isopropyl ether, methyl ethyl ketone, n-hexane, phenol and its derivatives, tetrahydrofuran, and toluene; and mixtures of such solvents. For example include the use of a buffer to breakdown mucans in saliva so that a target analyte, such as tacrolimus, is available to bind to an antibody, wherein the antibody is functionalized to a surface to effect separation.

Biological treatment of a sample may employ methods to separate biologicals based on selective binding of amino acid and nucleic acid sequences, based on chemical affinity and shape to other chemical or biological substances. Examples include the binding of the spike protein of the SARS-COV 2 virus by an antibody, or chemical bonding a segment of the SARS-COV 2 virus RNA by its compliment nucleic acid sequence, wherein the antibody and compliment are functionalized to a surface or magnetic bead to effect separation.

Physical treatment for effecting mutual separation may involve passage of the sample through a filter. Suitable filters comprise porous substrates such as paper, coated paper, paper fibers, polymer, polymer fibers, mixed paper and polymer fibers, cellulose acetate, glass wool, cotton, diatomite, porous glass, sintered glass, zirconia-stabilized silica, derivatized silica-based matrices, sol-gels, and derivatized sol-gels. They may also comprise a supported membrane covered with separation materials, such as the silica gels, zirconia-stabilized silica, derivatized silica-based matrices, sol-gels, derivatized sol-gels, glass beads, long-chain alkane particles, derivatized long-chain alkane particles, polymers, derivatized polymers, functionalized membranes, alumina, polystyrene, dendrimers, immobilized crown ethers, and ion-exchange resins.

Flow assay strips are based on thin layer chromatography, in which a solvent carries components across a surface that separates the components, based on chemical or physical characteristics, as they flow, driven by capillary action. Sandwich type LFAS strips contain the following components on a thin strip support structure: (a) a sample pad for sample deposition, (b) a conjugate pad containing detectable probes functionalized with a first binding agent to bind to the target analyte, (c) a test line functionalized with a second binding agent that also binds the target analyte forming a chemical, biochemical, or biological sandwich structure, (d) a control line functionalized with a third binding agent to bind excess probes, and (e) a wicking pad.

The probes referred to herein will desirably be composed of a central metal core, coated with a reporter molecule, encapsulated in a silica shell, and functionalized with a target analyte-specific binding agent. The metal core will, in general, be of the form selected from solid metal, or metal coated material, mono sized or distributed sized spheres, oblate spheroids, star and urchin shaped, pitted metal spheres, in solution as isolated particles, clusters, aggregates, ring or tube structures, or other appropriately sized structures. Specific examples include silver colloids, aggregates of gold colloids, gold-coated polystyrene spheres, and gold-coated magnetic iron beads.

The probe metal should, in any event, be readily coated with a reporter molecule that attaches to the metal surface through chemical bond or physical interaction, including covalent, ionic, hydrogen bonds, or van der Waals or electrostatic forces, between the metal and the reporter molecule. Ideally, the reporter molecule will form a chemical bond with the metal surface and have a spectral wavelength absorption, so as to be measured as intensity by a smartphone camera and an App, herein at the test line and control line of an FAS.

Dyes often satisfy these requirements, such as Alizarin, Allure Red, Brilliant Green, Cresol Red, Crystal Violet, Phenolphthalein, Pigment Blue, Fluorescein, Methyl Orange, Nile Blue, Phenol Red, Sunset Orange, and Thiazol Yellow.

A silica shell is used to both stabilize the dye coating so that it does not degrade over time, and provide a surface to attach a binding agent. This can be accomplished by adding the dye-coated metal nanoparticles to a solution of a silicate, and allowing the reaction to occur over several days, after which a silica shell with a thickness of 3-5 nm is formed. Ideally sodium silicate is used to form a coating in 5 days.

Functionalizing the silica surface with a suitable target analyte-specific binding agent; i.e., a chemical, biochemical, or biological substance, will in general include the formation of a chemical bonds or physical interactions, such as covalent, ionic, or hydrogen bonding, or by van der Waals or electrostatic forces between charged, polar, hydrophobic, or hydrophilic chemical groups on the surface of the “at least one” analyte and binding agent.

The flow assay strips are generally cut from a sheet of nitrocellulose with semi absorbent and highly absorbent strips glued across the top and bottom of the sheet, representing the sample and wicking pads, respectively. The conjugate probes, test line and control line binding agents are added as lines across the sheet, often using inkjet printer technology. Important parameters include the concentration and spacing of the probes, test and control line binding agents. A 30 cm×6 cm sheet could produce approximately 75 flow assay strips (0.4 cm by 6 cm).

### General Example A

A patient takes a treatment drug every day that is necessary to maintain his or her health. Instead of going to a clinic and providing a body fluid sample, such as a blood, saliva or urine sample, once per week or month as required to monitor health or establish compliance, the patient performs an at-home test using a blood, saliva or urine sample, a sample test kit with lateral flow assay strip (LFAS), and a smartphone with an analysis App to quantify the drug concentration and send the data to their physician, and possibly their healthcare provider. In this example a metabolite of the drug is measured, otherwise known as the target analyte. In other cases involving drug analysis, the target analyte may be the drug as its normal molecular structure, sometimes referred to as the parent drug.

More specifically, a urine sample is collected in a cup, a part of which is added to a vial containing a chemical reagent used to promote mutual separation of the target analyte from other chemicals and biologicals in the urine. For this case, the chemical reagent is composed of polar and non-polar chemicals, in which the latter solvates the target analyte. It may also be advantageous to physically separate the target analyte from other interferents, such as large molecules. For this case, a filter is used to pass only the small molecules, specifically the target analyte, while retaining larger molecules, such as particulate matter in urine.

After separation of the target analyte, a predefined portion of the separated sample is added to the sample pad of a LFAS using a transfer device. For this case, the sample transfer device is a disposable plastic pipette, which is used to transfer 3 drops to the LFAS sample. The sample flows across the LFAS driven by capillary action.

The LFAS is of standard design containing a sample addition pad, a conjugate pad, a test line, a control line, and a wicking pad, all of which are on the surface of a suitable support, such as nitrocellulose, paper, plastic, or a combination of these materials. Design factors include the conjugate pad, test and control line binding agent types and concentrations. In some cases, the LFAS will be contained in a plastic cassette or cardboard holder, which has a sample addition port, and an open section to view the test and control lines.

The conjugate pad is composed of signal-enhancing probes, each with a first binging agent that binds to the target analyte as it flows across the pad. For this case, the signal-enhancing probes include nanoscale gold particles coated with dye molecules, encapsulated in silica, and functionalized with an antibody specific to the drug metabolite.

The test line is composed of a second binding agent that binds the target analyte at the test line. Most often the test line binds to a different site of the target analyte, such that, if present, the target analyte plus signal-enhancing probes will be bound to the test line forming a sandwich structure with the target analyte between the probe binding agent and the test line binding agent. The control line is composed of a third binding agent that binds any excess probes not bound to the target analyte as the sample flows past the control line.

The LFAS is then placed in a holder that supports and aligns a smartphone camera above the LFAS. The App on the smartphone is used to measure and quantify the intensity of the LFAS test line, and if appropriate the control line.

Successful quantitative analysis is defined by a number of parameters, such as the volume of the urine sample added to the reagent vial, the volume of sample used in either the chemical of physical means of separation, the concentration of the probes on the conjugate pad, the number of binding sites available at the test line, and in some cases the concentration of binding sites available at the control line. In practice, these parameters are designed based on the expected target analyte concentration range, and a calibration curve is prepared for that range using a series of known target analyte concentration samples.

Calibration curves may take many shapes, but are most often described by a straight line, a Langmuir or an Avrami equation. The Langmuir equation describes a linear growth followed by an exponential decay of a measured signal limited by the number of binding sites for a target analyte. The Avrami equation describes an exponential growth followed by an exponential decay of a signal, the decay, again limited by the number of binding sites for a target analyte.

The equation is chosen based on these parameters, as well as the intensity measured at the test line without a sample, and sometimes in conjunction with the intensity measured at the control line. The control line is used as an intensity reference in some cases, as it may indicate the initial concentration of the probes at the conjugate pad.

For example, the measured intensity at the test line may be divided by the control line intensity, or it may be divided by the test line plus the control line intensities. In all cases, the intensity measured using the smartphone can be the absorbance, absorption, optical density, reflectance, reflection or one of or all of the red, green, blue color values of the LFAS lines, as may be required for quantitation. The test and control line, the latter when needed, are measured, ideally, at specified times, such as 5 minutes after all of the signal-enhancing probes have traversed the control line.

The time it takes for the sample to flow from the sample pad to the wicking pad will typically range from 1 to 30 minutes, depending on the sample volume and chemical, biochemical, or biological composition, first, second, and third binding agent composition and LFAS composition and design.

To ensure accurate quantitation, a QR code or barcode corresponding to a manufacturer's quality controlled LFAS production run is included on each sample kit, such that when scanned by the smartphone, the App will automatically set the constants for the appropriate quantitation equation. In the case of quantifying a drug in a urine sample, a straight line equation. Y=AX+B, may be used. Where Y is the concentration of the drug. X is the intensity of the probe-drug-test line sandwich structure measured by the smartphone camera, and A and B are the constants supplied by the QR code or barcode that are used calculate the concentration of the drug in urine.

The analysis of the drug concentration is automatically displayed on the smartphone as either 1) specific terms, such as ng/cc, ng/mL, mg/L, parts-per-million, 2) general terms such as low, good and high, or 3) both; and sent to the patient's physician manually or automatically, including, but not limited to by text, email, a phone call, or other means available on the smartphone. The measurement result can also be manually or automatically sent to the patient's healthcare provider and uploaded to other repositories of medical records.

Furthermore, analysis of a body fluid, using the presented probes, LFAS, and a smartphone for quantitation of a drug, could be used by a physician, physician's assistant, nurse or the like to perform a point-of-care analysis of a patient or employee to determine compliance, adjust prescribed dosage, determine severity of overdose or the like of a person in an office, a waiting room, an emergency room, or an ambulance.

### General Example B

People planning on entering a confined place with many people perform a self-test to determine if they are infected by a communicable pathogen beforehand. Examples of such places include indoor theaters, sports arenas, airplanes, trains, subways and buses, as well as healthcare facilities, such as a hospitals, clinics, and retirement homes, the latter facilities having occupants with greater risk of infection, illness and death. Many respiratory infections, such as coronaviruses, influenza, M. tuberculosis and Streptococcal pneumonia are spread by sneezing, coughing and talking. Before entering such places, a person is required to perform an at-home or point-of-care test to determine if they are infected with the pathogen of concern at the time. For example, in 2020 to 2022 the pathogen of concern was the SARS-CoV-2 virus. In the case of hospitals, methicillin-resistant Staphylococcus aureus (MRSA) and Streptococcal pneumonia have long been pathogens of concern. In both environments, it may be okay for a person to enter such a confined space if they are infected, but not contagious, hence quantitative analysis is required.

A person performs an at-home self-test using a nasal mucus, nasopharyngeal, saliva, or throat sputum sample, a sample test kit with a LFAS, and their smartphone to quantify the pathogen-of-interest and uploads the data to a website, cloud site, or similar appropriate site requiring the screened results.

More specifically, a nasopharyngeal sample is obtained by swabbing both nasal cavities in a circular motion 10 to 20 times to achieve the desired quantitative accuracy. In some cases, sample collection using a nasal lavage may be preferred. The swab is added to a soft-sided plastic vial with a mucolytic reagent, such as N-acetyl-L-cysteine (NALC) to break down the mucans freeing the pathogen for analysis.

After allowing time for the reagent to perform its function, such as 5 or 10 minutes, the swab is removed while squeezing it with the walls of the soft-sided plastic vial, to release the reagent plus sample into the vial.

A predefined portion of the reagent plus sample solution is then added to a LFAS as described in General Example A, and may or may not be encased in a cassette.

The test line and control line employ binding agents to bind the target analyte and excess probes, respectively, as described in General Example A. The sample added to the sample pad, flows across the LFAS to the wicking pad, and is then placed, aligned, measured, and quantified using a smartphone camera and App as described in General Example A. Quantitation, however, may be reported in specific numerical terms such as a viral load and colony forming units, or in general terms such as contagious and noncontagious, as is appropriate to the measured pathogen and the information needed. The results are then uploaded to the site requiring the screened results, or presented to an attendant at the entrance to the confined space.

### General Example C

In some of the cases described in General Example B, the confined place with many people, such as a sports arena, hospital and pharmacy, may have a drive through set-up and the like in which the sample is collected by the person needing to be tested and they give the sample to another person, certified to perform a point-of-care test. The certified person then uses a corporate smartphone to perform the test, for example, that allows sharing the data with the individual and the attendants of the confined space.

It is realized of course that during an influenza or coronavirus outbreak or pandemic, that it would be most expedient to measure LFAS collected from multiple patients using a high-throughput device capable of rapidly preparing and measuring the test line, and as needed the control line of numerous samples, such as an XY positioning stage that holds multiple LFAS in cassettes and a rotating carousel, that allows continuous sample addition, LFAS analysis, and disposal. Such expediency could aid the quarantine process, and potentially decrease person-to-person infections.

As mentioned in General Example B, many respiratory infections, such as coronaviruses, influenza, M. tuberculosis and Streptococcal pneumonia are spread by sneezing, coughing and talking. As an alternative to nasal and salvia collection, collection of a breath sample may be more suitable.

The person is instructed to take a deep breath and blows into a balloon connected to a valve until the balloon fills a rigid container, and then they close the valve. The valve is then connected to a vial containing probes and opened until the balloon collapses, and then the valve is closed. The vial is added to a high-throughput sample preparation and measurement device as described above. For this case, the LFAS does not have a conjugate pad with probes located between the sample pad and the test line. Otherwise the LFAS is of standard design,

### General Example D

In another mode of use of the instant method, a target analyte-containing sample obtained from a surface, representative of surfaces associated with hospitals, local medical clinics, laboratories, food production equipment, and the like, is examined to detect potential contamination by pathogens. Example pathogens include those listed in Example B, as well as Escherichia coli (0157: H7), Listeria monocytogenes, and Salmonella enterica, the latter three being a concern in the food production industry.

A non-cotton swab will generally be preferred to collect a sample of a predefined surface area. The swab is then added to a transfer container, such as a vial and tube, of solution, from which a predefined volume is delivered to the sample deposition point of a lateral flow assay strip. In cases when the amount of the pathogen on the surface is on the order of 1 to 100 colony forming units per square centimeter (CFU/cm²), the container of the solution will include a medium that grows the pathogen to 103 to 105 CFU/mL, typically with the aid of heat, to allow detection by the present invention, realizing that current technology requires 106 to 109 CFU/mL for detection. In such cases, the signal-enhancing probes may be then added to the container and allowed to bind to the grown pathogens. A separation step may then be required to isolate the probe-bound pathogens prior to addition to the LFAS.

This may include centrifugation, filtration and solvent extraction. In some cases, such as higher initial pathogen concentrations, the analyte sample could be added to a LFAS that has probes immobilized on a conjugate pad between the sample addition pad and the test line. In any case the appropriate LFAS and the test and control lines are measured as described above. Since the surface area, amount of transfer solution, if used, and growth solution, and antibody probes used in the present procedures would generally be known, that information can be used to quantify the pathogens detected in terms of surface area (e.g. 1 CFU/cm²), again using the appropriately prepared calibration samples as described above. It is also reasonable to consider that a LFAS can be designed to detect more than one target analyte, representing more than one pathogen, such as the three pathogens listed in this General Example. In such cases a cassette may desirably be a multi-strip holder that contains several flow assay strips in parallel, one per target pathogen, in which the sample is added to the sample pads on each of the strips, each with their appropriate conjugate pad probes, and test and control line binding agents. Alternatively, several LFAS strips could be placed into a sample holder, such as a cup, and the sample flows upward along the strips by capillary action. Such vertical flow assay strips (VFAS), would then be removed for quantitation.

In other cases a multi sample LFAS could be used to measure more than one structural or molecular subunit of the same pathogen to improve the specificity of the LFAS, such as the SARS CoV-2 spike and nucleocapsid proteins described in General Example B.

### General Example E

A patient describes a series of symptoms they are experiencing to a physician, who suspects are due to one or more particular genetic diseases. Such diseases include amyotrophic lateral sclerosis, cystic fibrosis, Down syndrome, muscular dystrophy, hemophilia, and Huntington disease, and Parkinson's disease, among others. While standard practice may be to have a sample collected and sent to a lab, the turnaround time may be as much as a month. This time delay may be unacceptable to the patient, especially if the answer dictates how much time they have to live. The instant invention can address the delay and the patient's need to know, wherein the physician performs a point-of-care analysis using for example a blood sample, wherein quantitation can estimate the stage of the disease and in turn the patient's longevity.

Based on the analysis the physician collects a sample, such as blood or urine from the patient, and uses a LFAS to detect and quantify the disease specific biomarker as the target analyte using his smartphone equipped with the appropriate App. Most often the target analyte will be a genetic sequence, however, other disease biomarkers may be used, such as peptides, aptamers, and antibodies. As in the previous general examples, the LFAS will have a standard design with or without the conjugate, but the binding agents will preferably be the complement to the target analyte, such as a genetic sequence and antigen, as the case may be.

### BRIEF DESCRIPTION OF THE DRAWINGS

FIG. 1 is a diagrammatic illustration of a lateral flow assay strip (LFAS) design consisting of a sample pad, a conjugate pad, a test line, a control line and a wicking pad on a support structure; showing the addition of a target analyte, a probe on the conjugate pad, a probe bound to the target analyte, which in turn is bound to the control line binding agent forming a sandwich structure, and excess probes bound to the control line binding agent.

FIG. 2 is a diagrammatic illustration showing components of the apparatus embodying the invention, depicting a smartphone on top of a holder with the smartphone camera aligned to measure the test line of a LFAS in a cassette, including cassette details, and a device to hold and manipulate multiple LFAS and cassettes.

FIG. 3 is a diagrammatic illustration showing components of apparatus embodying the invention, depicting a kit containing a LFAS, sample collection and transfer devices, reagents, filters, and smartphone readable codes.

FIG. 4 is a diagrammatic illustration showing components of apparatus embodying the invention, depicting in a series of steps comprising a method embodying the invention, for measuring a series of samples with known target analyte concentrations on a series of LFAS to produce a concentration calibration curve and a QR code on a kit by a manufacturer, which can be read by a customer using a smartphone to upload the calibration curve to the smartphone App to be used to measure unknown samples.

FIG. 5 is a diagrammatic illustration showing components of apparatus embodying the invention, depicting in a series of steps comprising a method embodying the invention, for measuring the opioid treatment drug, buprenorphine, in urine by a physician of a patient at the point-of-care.

FIG. 6 is a diagrammatic illustration showing components of apparatus embodying the invention, depicting in a series of steps comprising a method embodying the invention, for measuring the SARS-CoV-2 virus in a nasopharyngeal sample collected by a person at-home to determine their infectious level with the ability to send the data to others or databases.

FIG. 7 is a diagrammatic illustration showing components of apparatus embodying the invention, depicting in a series of steps comprising a method embodying the invention, for measuring foodborne pathogens on food production equipment with the ability to send the data to others or databases.

FIG. 8 is a plot of the absorbance for six samples of 0, 2.5, 5, 7.5, 10, and 12.5 ng/ml tacrolimus spiked in saliva measured by a Smartphone Camera and App.

FIG. 9 is a diagrammatic illustration showing the basic structure of a SARS CoV-2 virion, and the spike protein of the virion.

FIG. 10 is A) a photograph of 4 cassettes prepared using 4 purchased SARS-CoV-2 samples with PCR Ct values as indicated, and 1 cassette prepared using saliva without the virus; B) a plot of reflectance values of the LFA test lines for 4 samples and a saliva blank fit with an Avrami equation; and C) a plot of reflectance values of the LFA test lines for the 16 diluted samples calculated using Equation 2, shown as a black line.

### DETAILED DESCRIPTION OF THE PREFERRED AND ILLUSTRATED EMBODIMENTS

As indicated above, the present invention provides a novel method and apparatus to detect, identify, quantify, and analyze target analytes in test samples that bind first to an analyte-specific binding agent attached to a signal-enhancing probe and bind second to an analyte-specific binding agent attached to a flow assay strip, which provides exceptional selectivity with unexpected signal enhancement. The former provides identification, while the latter provides the ability to use a smartphone equipped with a camera and an App to perform detection, quantitation, and analysis with speed, ease-of use, at-home, point-of-care, or point-of-need.

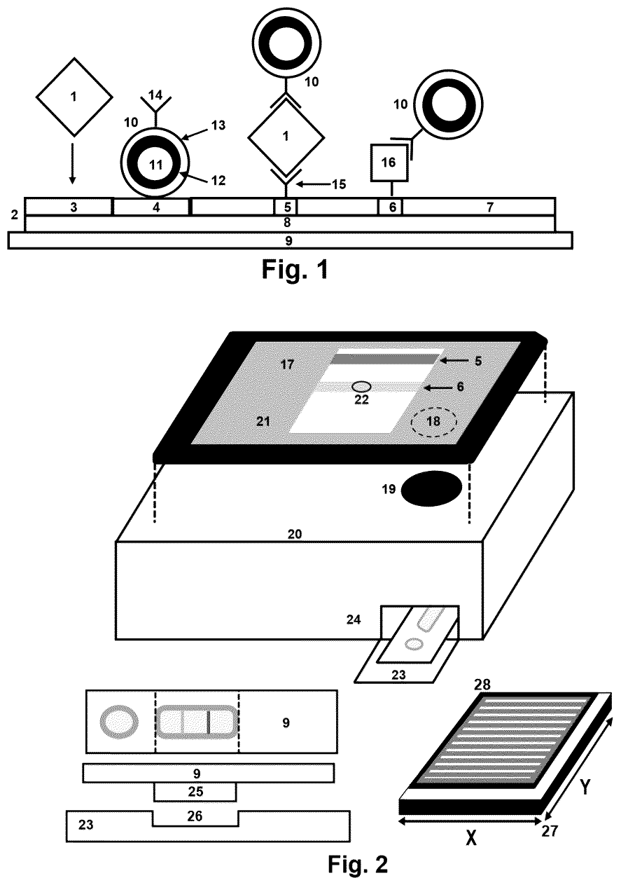

Turning now in detail to FIG. 1, therein illustrated, is a side-view diagram showing a target analyte 1 being added to a lateral flow assay strip 2 composed of a sample pad 3, a conjugate pad 4, a test line 5, a control line 6 and a wicking pad 7, all on a suitable support structure 8. In some cases the LFAS is encased in a plastic or cardboard cassette 9. The conjugate pad 4 contains signal-enhancing probes 10 composed of a metal core 11 surrounded by reporter molecules of one type 12, encapsulated in silica 13, and functionalized with a first binding agent 14. The test line 5 contains a second binding agent 15 that may or may not be the same as the probe binding agent 14, while the control line 6 contains a third binding agent 16 that is different from the other two. In use the target analyte 1 in a solution is added to the sample pad 3, where it flows, driven by capillary action, first, across the conjugate pad 4 containing the signal-enhancing probes 10, which become mobile, mix and bind to the target analytes 1, if present, second, the probe-target analyte conjugates flow across the test line 5 functionalized with the second analyte-specific binding agent 15, wherein the conjugates bind if they have bound target analytes forming a sandwich structure, and third, the sample continues to flow towards the wicking pad 7, passing across the control line 6 functionalized with the third binding agent 16 specific to the probes, wherein unconjugated probes bind. Detection of a signal at the test line 5 by a smartphone using an appropriate App indicates positive detection of the target analyte 1 with extreme sensitivity, selectivity, and speed, while detection of a signal at the control line 6 indicates that the signal-enhancing probes and the LFA are working properly.

The smartphone App is used to quantify the intensity of the signal-enhancing probes at the test and control line in conjunction with a calibration concentration curve to further analyze the measurement in terms of the amount of target analytes. It is of course realized that a vertical flow assay strip, employing the same components, is most often used by dipping the sample pad end of the strip into a solution containing the sample with the target analyte.

FIG. 2 illustrates the apparatus embodying the invention, a smartphone 17 with a camera 18 that sits over a hole 19 in a smartphone-to-FAS holder 20, and points at the LFAS most often contained in a cassette 9 (top view) inside the holder 20. The App on the smartphone 17 is used to view a live image 21 using the camera 18 to allow manual or automated expansion and alignment of the test line 5 and, as needed, the control line 6, above the camera measurement point using an alignment feature 22, such as a circle, square, rectangle, bullseye, cross hairs, or the like.

The cassette 9 is placed in a tray 23 that slides through a side port 24 in the holder 20, which is designed to align the test line with the center of the camera image.

In cases that require measurement of both the test and control lines, the bottom of the cassette 9 will have a rectangular alignment plate 25, that is centered on the midpoint between the lines to ensure alignment of either the test line or the control line, depending on the direction that the cassette is placed into the tray 23 that will have a recessed rectangle 26 that matches the alignment plate.

In some cases, the tray 23 positioning is controlled by the smartphone App. In other cases, the holder 20 will contain a one axis, X, or two axis, XY, positioning stage 27 that holds multiple LFAS or individual cassettes in a single container 28.

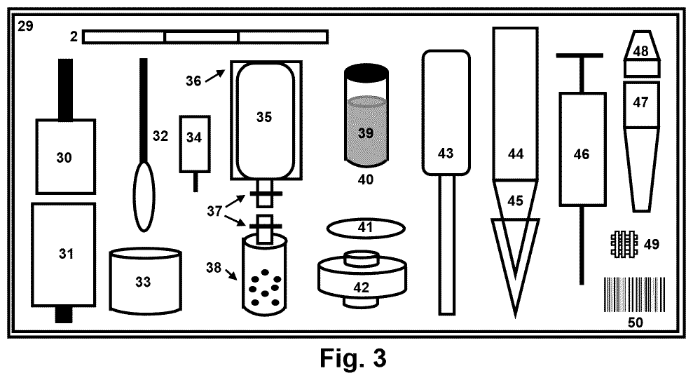

FIG. 3 illustrates a kit that includes the essential components of the apparatus of the present invention, contained in a case or pouch 29 of suitable construction. The contents of each kit will of course correspond to the application for which it is intended, but the main components are a lateral flow assay strip 2, a sample collecting device, and a sample transfer device. Sample collection devices include a swab 30, flat, barrel, or oblong shaped 32 with or without a compression sleeve or barrel 31, for collecting surface, solution, saliva, or nasopharyngeal samples, a cup 33 for collecting a urine or drooled saliva, or a lancet 34 for finger prick collection of blood, sample, or a balloon 35 within a rigid box 36 to define the volume, and a valve 37 to seal a breath sample inside prior to transfer, such as into a vial containing signal-enhancing probes 38. Sample preparation devices may include the use of a reagent 39, a reagent bottle 40, or replaceable filters 41 with a filter holder 42. Sample transfer devices include a pipette 43, an auto-pipetter 44 with disposable tips 45, and a syringe 46, some of which may be calibrated so that the sample volume added to a reagent or a LFA strip can be controlled.

In some cases, a device may be used for one or more of these functions. For example, a swab containing a collected sample could be inserted into a soft sided centrifuge tube 47 that a) contains a reagent to break down the sample to release the target analyte, and b) has an eye dropper cap 48 that can be used to deposit a specific number of drops on the sample pad of a LFAS.

For some applications, the kit may contain a multiplicity of components, such as twelve LFA strips, twelve disposable pipettes or twelve auto pipette tips, and the like.

In addition, other reagents in other containers may be necessary or desirable to a) aid in the collection of samples, such as a water or lavage buffer to collect nasal mucus or the like, b) digest samples, using a reagent such as NALC, c) liquefy samples, d) degrade biologicals, using an acid such as acetic acid, and e) wash away unbound chemicals on the LFA after the binding step, such as a chase buffer. It will be appreciated that containers for the latter solutions will be of suitable size and construction to best accommodate their contents. It will also be appreciated that the containers will advantageously have associated means for employing their contents, such means being integral with the container (e.g., an eyedropper cap 48), or separate therefrom, but adapted for use therewith (e.g., a pipette 43, a syringe 46, or the like).

A primary function of pipettes 43, a syringe 46, and like components of course is to transfer liquids and dispense samples onto the LFAs; they may for example be used to dispense water or other liquid onto a surface, to collect the possible pathogens present, or to introduce a sample onto the LFAs. Pipettes 43, a syringe 46, and filter holders 42 and replaceable filters 41 included in the kit, will desirably be constructed to connect to the corresponding sample dispensing components. A plunger-mounted swab 30 may be used to collect saliva or throat sputum, and its associated compression sleeve or barrel 31 may be used to discharge the sample into a sample container, such as a vial 39, with or without a reagent. A filter holder 42 and filters 41, may be employed in either collecting or transferring a sample, as required, and additional vials will be supplied, as necessary, for sample collection, transfer, or mixing; even a blender (not shown) may be included in the kit for blending a sample, such as food, with an extracting reagent, or small centrifuge may be included for separating serum from red blood cells, as may be necessary. For most applications, the case or pouch 29 will have a QR code 49 or barcode 50 on the outer surface unique to the production run.

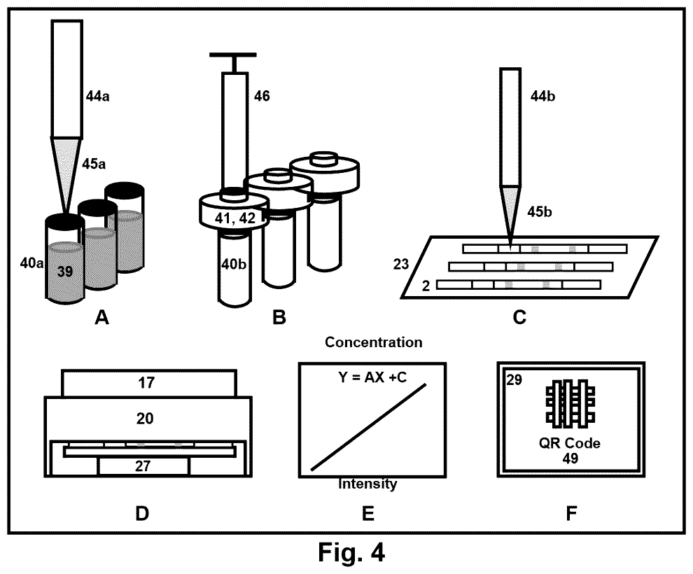

FIG. 4 shows the basic steps that a manufacturer would perform to produce an at-home, point-of-care, or point-of-need calibration curve for a new target analyte, and for every production batch of LFAS. The following example describes the measurement of the immunosuppressant drug, tacrolimus, in saliva. The basic approach can be applied to a wide variety of target analytes and sample media. Step A shows the addition of a series of 0.1 mL saliva samples containing tacrolimus using an auto pipetter 44 with disposable tips 45 into a series of vials 40 containing a 0.5 mL of reagent 39 used to break down the mucins to free tacrolimus for binding. While three vials are shown to represent 3 samples of different concentration, in practice, hundreds to thousands of samples will be prepared and measured for a new drug in a new sample medium. Step B shows a series of syringes 46 being used to pass a treated sample through a series of filters 41 in filter holders 42 into a series of vials 40, to remove the degraded mucans from the samples. Step C shows 100 microliters being deposited onto a series of flow assay strips 2 that are located on a multi-strip tray 23. It of course is realized that a series of cassettes 9 (FIG. 1) could equally be used for developing a calibration concentration curve. Step D shows the tray 23 placed on an XY positioning stage 27, located inside the smartphone-to-FAS holder 20, where the intensities of the test lines of each FAS is measured using the smartphone 17 camera 18 (FIG. 2) and App. In general the XY positioning stage would only need to move along one axis performing measurements at the test lines as illustrated in the figure. However, if a several trays are used, positioning along the other axis would of course be required. In such cases, the holder 20, would be considerably larger than suggested by FIG. 4. Furthermore, it may be advantageous to move the smartphone 17 instead of the trays 23. Step E shows the resultant concentration calibration curve developed using the measured intensities and known concentrations of the series of samples. Step F shows a printed QR code symbol 49 on a kit 29, which when scanned by a smartphone with the App of this invention will automatically load the concentration calibration curve to be used by this kit 29. Although not part of this patent application, as stated above, numerous samples of known concentration, as well as unknown concentration, would be used to develop the concentration curve, along with multivariate statistics to provide a concentration calibration curve that meets the standards of the appropriate industry.

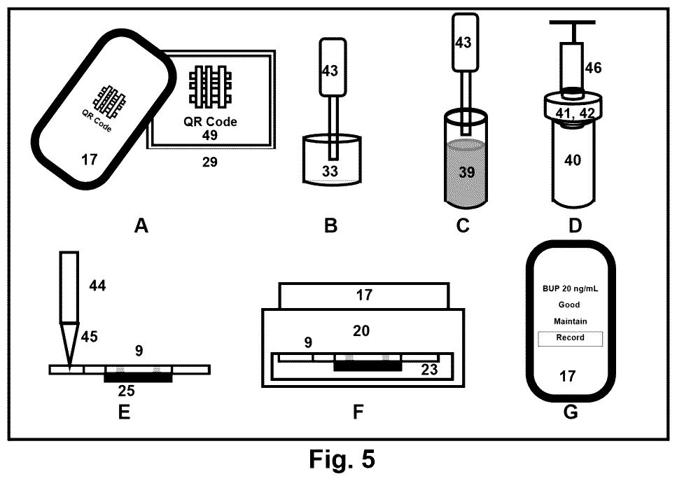

FIG. 5 shows the basic steps that a physician would perform to provide point-of-care assessment of a patient taking an opioid treatment drug, such as buprenorphine, using a urine sample and a smartphone equipped with the appropriate App. The basic approach can be applied to a wide variety of target analytes and sample media. Step A shows the physician scanning the QR code 49 on the sample kit 29 using a smartphone 17 to load the appropriate calibration curve for buprenorphine in urine to the App. Step B shows drawing 1 mL of urine from a sample cup 33 into a pipette 43, potentially containing the drug (i.e., the target analyte). Step C shows transferring the 1 mL drawn sample, using the pipette 43, to a vial 40 containing a reagent 39, such as a buffer that neutralizes the solution. Step D shows using a syringe 46 to passing the treated sample through a filter 41 in a filter holder 42 into a vial 40 to remove any particulate matter in the treated sample. Step E shows the addition of 200 microliters of the particle free treated sample using an auto pipetter 44 with disposable tips 45 into to a LFAS cassette 9 that contains an alignment plate 25 on the bottom. Step F shows the inserted cassette 9 in a tray 23 that has a slot for the alignment plate 25, within the holder 20. For this analysis the test line of the cassette is inserted test line end first, and measured, then it is inserted control line end first and measured. Step G shows the calculated concentration using, in this case, a calibration curve that uses the ratio of the test line intensity divided by the sum of the test and control line intensities. The smartphone can also provide additional analysis based on the buprenorphine concentration, as well as the ability to send the information to the physician's database and the patient's medical records.

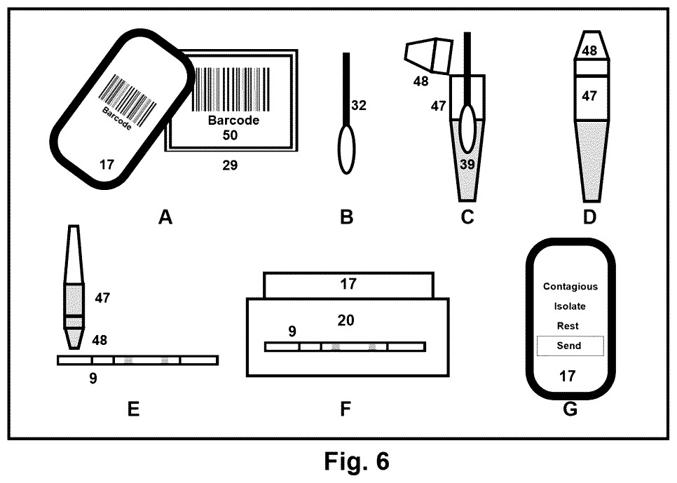

FIG. 6 shows the basic steps that a person would perform at-home to assess their level of infection by a virus, such as the SASR-CoV-2 virus, using a nasopharyngeal sample and a smartphone equipped with the appropriate App. The basic approach can be applied to a wide variety of target analytes and sample media. Step A shows the person scanning the barcode 49 on the sample kit 29 using a smartphone 17 to load the appropriate calibration curve for SASR-CoV-2 virus in a nasopharyngeal sample. Step B shows a swab 32 that is used to collect the sample by swabbing the walls of both nostrils, potentially containing the pathogen (i.e., the target analyte). Step C shows inserting the sample into a reagent 39 contained in a soft plastic vial 47 that has a connected dropper cap 48. Step D shows the vial with the cap on after squeezing the sides of the vial while the swab is removed to retain the sample. Step E shows the addition of 3 drops from the soft plastic vial 47 with a dropper cap 48 onto a LFAS cassette 9. Step F shows the inserted cassette 9 in the holder 20. For this analysis only the test line is measured. Step G shows the status of the person's infection with suggested safety measures, and the ability to send results to people and health agencies.

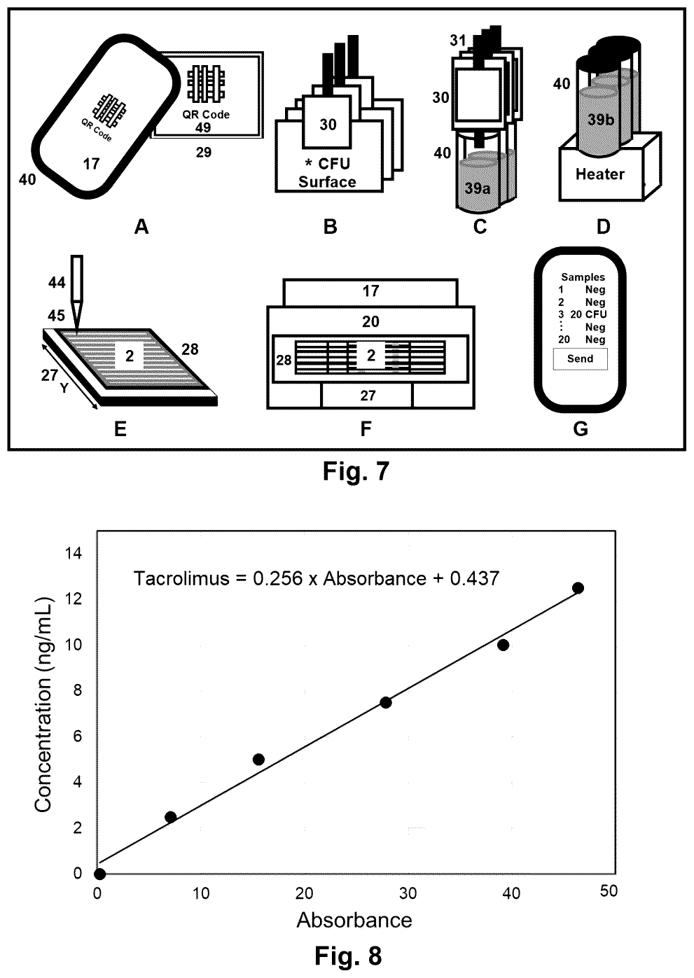

FIG. 7 shows the steps for measuring a pathogen, such as a L. monocytogenes, E. coli (O157: H7), or Salmonella enterica on a surface, using a swabbed sample and a smartphone equipped with the appropriate App. The basic approach can be applied to a wide variety of target pathogenic analytes and sample media, including food samples. Step A shows using a smartphone 17 to load the appropriate calibration curve for the target pathogen on a surface by scanning the QR code 49 on the sample kit 29. Step B shows a series of swabs 30 with broad widths used to wipe a series of specified surfaces, with specified dimensions, such as 10 cm by 10 cm, potentially containing the pathogen (i.e., the target analyte) on a food production equipment surfaces. Step C shows the swabs being inserted into a corresponding series of compression sleeves 31 containing a reagent 39a to extract the pathogens, if present, and then being expelled into a corresponding series of vials 40 containing a second reagent 39b to grow the target pathogen, often referred to as a broth. Step D shows the series of vials 40 on a heater, not supplied with the kit, used to grow the pathogen of interest to the point of detection. Step E shows the deposition of the first of a series of samples, drawn by an auto pipetter 44 from the growth medium, onto a container 28 of series of LFA strips 2 using disposable tips 45. It of course is realized that a series of cassettes 9 could equally be used for performing these measurements. The deposition of samples onto the conjugate pad, are preferably, performed automatically using a positioning stage 27 that moves the LFA strips along one axis from conjugate pad to conjugate pad. Step F shows the tray 23 placed on a one axis positioning stage 27, located inside the holder 20, that aligns and measures the test line of each LFA strip using the smartphone 17 camera 18 and App. Step G shows the results for each sample as negative or with a concentration, in the case of a foodborne pathogen, most often as colony forming units (CFU), and the ability to send results to appropriate people, databases and health agencies.

It some cases the reagent vial 40 might contain a generic growth medium reagent that grows several pathogens, and the sample drawn by pipetter 44 may deposit some of the sample on separate LFA strips 2 contained on the tray 23 designed to detect each target pathogen as described in this example. It should be of course realized that the present invention employing methods equivalent to those described in these examples could be used to measure other target analytes, such as antibodies, disease biomarkers, or drugs in body fluids or on surfaces.

FIG. 8 shows a plot of the absorbance for six samples of 0, 2.5, 5, 7.5, 10, and 12.5 ng/ml tacrolimus spiked in saliva measured by a Smartphone Camera and App. The data was fit with a straight line equation, viz. Tacrolimus=0.256×Absorbance+0.437.

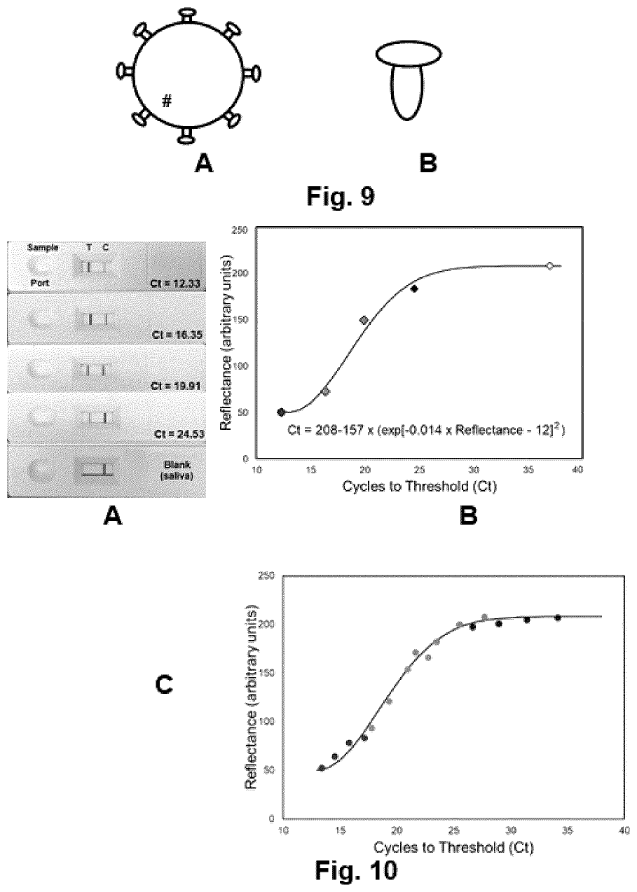

FIG. 9 shows a diagrammatic illustration showing the basic structure of A) a SARS CoV-2 virion, and B) the spike protein of the virion.

FIG. 10 shows A) a photograph of 4 cassettes to which four purchased saliva samples with PCR Ct values of 12.33, 16.35, 19.91, and 24.53 were added, along with 1 cassette to which a saliva sample devoid of SARS-CoV-2 was added, B) a plot of the reflectance values of the LFA test lines for the 5 samples measured by a Smartphone Camera and App (diamonds), fit with an Avrami equation, Equation 2 shown as a black line, to produce a calibration curve, and C) a plot of the reflectance for 4 samples each prepared from dilution of the 4 purchased samples measured by a Smartphone Camera and their corresponding Ct values calculated by the App using the calibration curve (circles).

### Exemplary of the Efficacy of the Present Invention are the Following Specific Examples:

### Example One

Detection of Tacrolimus in Saliva by a Smartphone with an App

Previously, it was noted that test lines of lateral flow assay strips, designed to measure tacrolimus at ng/mL concentrations by surface-enhanced Resonance Raman spectroscopy (SERRS, Farquharson et. al., J Anal Bioanal Tech, 2022), showed differences in intensity as a function of concentration visible to the naked eye. This was entirely unexpected, as the SERRS probes, consisting of synthesized gold nanoparticles, coated with a blue dye, as a reporter molecule, and functionalized with antibodies specific to tacrolimus, were designed to amplify Raman scattering by a minimum of 9-orders-of-magnitude, such that a laser-based Raman spectrometer could detect such low concentrations. Tests showed that a laboratory visible spectrometer could be used to measure the absorbance of the SERRS probes at the test line. Consequently, a simple apparatus was designed to determine if it a series of tacrolimus samples could be measured by a smartphone. The apparatus included a simple box to hold the smartphone above the cassette, with a hole in the top so that a camera could illuminate the sample (FIG. 2). A smartphone App was written to 1) magnify the test line image, 2) ensuring that the test line was centered, and 3) measure the test line absorbance. The following concentration series of tacrolimus in saliva was prepared: 2.5, 5, 7.5, 10, and 12.5 ng/mL. A pipette with an auto-volume setting was then used to deliver 100 microliters of each sample to a separate cassette with a lateral flow assay strip. The LFAS were of standard design consisting of 1) a conjugate pad containing the SERRS probes, 2) a test line functionalized with the tacrolimus antibodies, 3) a control line functionalized with goat anti-mouse IgG, and 4) a wicking pad, all on 5) a Nitrocellulose Support. The LFAS were contained in plastic cassettes that had a sample introduction port and an open section to view the test and control lines. The test line of each cassette was then measured using the smartphone holder, camera and App. A plot of the 6 sample measured absorbance versus the prepared concentrations showed excellent linearity for the concentration range typical used for transplant patients (FIG. 8, Table 1).

The calibration data was fit with a straight line, Equation 1, with multiplication and offset calibration constants of 0.2559 and 0.4371, respectively, defining the relationship between the measured absorbance and tacrolimus.

$$Tacrolimus = 0.2559 \times Absorbance + 0.4371 \qquad Equation 1$$

The relationship was tested by diluting a 12.5 ng/ml sample two times by 50% to produce three “unknowns”, 12.5, 6.25, and 3.125 ng/mL. The absorbances of the three unknown samples were then measured by the smartphone camera, and the App, and Equation 1 was used to calculate the corresponding tacrolimus concentrations. The calculated concentrations were close to the prepared concentrations with an average difference of 0.51 ng/ml (Table 2).

TABLE 1
Measured absorbance by a Smartphone App of the LFAS
test lines for 6 prepared tacrolimus samples.

| Prepared Tacrolimus | Test Line |
|---|---|
| in Saliva, ng/mL | Absorbance |
| 0 | 0.3 |
| 2.5 | 7.1 |
| 5 | 15.6 |
| 7.5 | 27.2 |
| 10 | 39.2 |
| 12.5 | 46.4 |

TABLE 2
Measured absorbance by the Smartphone and App of the LFAS test lines
for three prepared “unknown” samples and the corresponding
tacrolimus concentrations and errors calculated using Equation 1.

| | | Calculated | Actual | Error |
|---|---|---|---|---|
| “Unknown” | | Tacrolimus | Tacrolimus | (Actual − |
| Tacrolimus | Test Line | Concentration | Concentration | Calculated |
| Samples | Absorbance | (ng/mL) | (ng/mL) | ng/mL) |
| LFA 1 | 45.5 | 12.1 | 12.5 | 0.40 |
| LFA 2 | 20.7 | 5.7 | 6.25 | 0.55 |
| LFA 3 | 8.3 | 2.5 | 3.125 | 0.59 |
| | | | Average Error | 0.51 |

The following continuation of Example one represents intended measurements. Once a month, an organ transplant patient measures the level of tacrolimus in their saliva using the present invention, which includes a combination smartphone-cassette holder, an App on their smartphone, and a kit with a QR code on the cover, which contains a 6-month supply of LFAS, sample collection vials, reagent vials, and plastic pipettes (FIG. 3). The patient launches the App for tacrolimus analysis on the smartphone, and performs the following steps:
1) Scan the QR code on the kit using the smartphone to automatically update the calibration curve used by the App
2) Press “Instructions” to review the measurements steps as needed, which include:
3) Open the kit,
4) Collect a saliva sample by passive drooling into a vial,
5) Transfer 1 mL of the saliva sample, using a disposable plastic pipette, into a second vial containing a reagent to break down the saliva mucans freeing tacrolimus for analysis,
6) Press “App Timer 1” to indicate when mucan break down is sufficient (e.g. 5 minutes),
7) Transfer 3 drops, of the degraded sample to the LFAS cassette sample port using a disposable pipette,
8) Press “App Timer 2” to indicate when to measure the test line (e.g. 20 minutes),
9) Place the cassette inside the holder and the smartphone on the holder,
10) Press “View Cassette” to view the automatically expanded and aligned image of the test line,
11) Press “Measure” and read the results (calculated according to updated calibration curve), automatically displayed after the predetermined time, as a number of ng/ml tacrolimus in saliva, and if appropriate labelled as low, good, or high,
12) Press “Send” to send the results to patient's physician.

Since it is known that the tacrolimus concentration in the blood is modestly higher than in saliva, more accurate results may be obtained by replacing the saliva sample with a drop of blood. In this case, the steps might be as follows:
1-3) As above,
4) Collect a drop of blood using the spring-loaded, finger needle lancet provided (FIG. 3),
5) Add the drop to a reagent used to separate and precipitate blood from tacrolimus,
6) Draw the sample through a filter into a syringe to collect the tacrolimus in reagent (FIG. 3),
7) Remove the filter on the syringe and deposit 3 drops on the cassette port,
8-12) As above.

### Example Two

### Quantifying SARS CoV-2 in Saliva

Probes, as described in the Example One, were modified as follows: a red dye replaced the blue dye, and the tacrolimus antibody, was replaced with mouse monoclonal SARS-CoV-2 spike antibody selected to bind to the S2 subunit of the spike protein and the nucleocapsid protein (subunit) of the SARS-CoV-2 virion (FIG. 9). Lateral flow assay strips in cassettes were prepared with a conjugate pad containing the antibody functionalized probes, a test line containing rabbit polyclonal SARS-CoV-2 spike antibodies selected to bind to the S1 subunit of the spike protein, and a control line containing Protein A selected to bind excess probes that did not capture SARS-CoV-2 virions.

Four purchased saliva samples with PCR Ct values of 12.33, 16.35, 19.91, and 24.53, along with a saliva sample devoid of SARS-CoV-2, were added to 5 LFAS in cassettes used to prepare a calibration curve (FIG. 10A). A pipette with an auto-volume setting was used to deliver 100 microliters of each sample to the 5 LFAS cassettes. As in the previous example the individual cassettes were mounted below the camera of a smartphone using a holder. However, in this case an App was written to measure the reflectance instead of the absorbance by the camera at the test line. Upon adding the sample to the cassette, the user presses “Start” on the smartphone App, which uses the smartphone timer function to measure the reflectance at the test line 20 minutes later.

The Ct 12.33 sample was used to represent a test line saturated with probes and the lower limit of reflectance, while the sample without SARS-CoV-2 was used to represent a test line containing no probes and the upper limit of reflectance. The test line reflectances for these two samples were quantified using the smartphone App as 208 and 50, respectively. Judicious selection of the concentrations of the probes at the conjugate pad and the antibodies at the test line allowed maximizing the detection range for the spike protein and the nucleocapsid protein.

As in the previous example, a clear decrease in the visible intensity of the test lines is apparent for the 4 samples and saliva blank as the Ct values increase, representing a decrease in the viral load (FIG. 10A). A plot of the reflectance as a function of Ct values for the 4 samples and the blank saliva sample was fit with the Avrami equation (FIG. 10B, Equation 2, Avrami M, J Chem Phys, 1939), most often used to describe a system that has an initial exponential increase followed by an exponential decrease. For the present system, an exponential increase in antibody binding is expected at the test line as the sample concentration increases, followed by an exponential decrease as all the bonding sites become occupied.

$$Ct = 208 - 157 \times ( exp [ - 0.014 \times {( Reflectance - 12 )}^{2} ] ) \qquad Equation 2$$

Next, a sample set of 16 “unknowns” were prepared by diluting each of the 4 purchased samples 4 times in de-identified, pooled saliva by 50%. This dilution factor was chosen to mimic the factor of 2 nucleic acid replication achieved from one PCR cycle to the next. As before, the samples were prepared, added to cassettes, and the reflectance measured at the test line, but this time the smartphone was also used to calculate the Ct values using Equation 2 (FIG. 10C).

The following continuation of Example Two represents intended measurements. A person obtains a SARS-CoV-2 virus test kit from a store, clinic or by mail, to be used to determine if they are infected using the present invention at home. The kit includes a saliva collection swab, a plastic vial with a reagent, a LFAS cassette (FIG. 3, items 32, 47 and 48, and 2, respectively), and a simple smartphone-cassette holder (FIG. 2, item 20). The person scans the QR code on the kit with their smartphone, which downloads an App that provides the following step-by-step instructions to perform the test, inclusive of any App updates and calibration curves.
1) Press the instructions tab to review the measurements steps as needed, which include:
2) Open the kit,
3) Place the swab on the middle of your tongue and suck on the swab in a pulsating fashion for 30 seconds, use the App timer if necessary,
4) Insert the swab into the plastic buffer vial, which breaks down the saliva mucans, and stir the swab for 30 seconds, then set aside for 5 minutes, use the App timer,
5) Pinch the neck of the plastic vial walls against the swab to squeeze the solution out of the swab as the swab is pulled out,
6) Add the dropper cap to the vial and transfer 3 drops of the sample solution to the cassette sample port,
7) Start a second App timer to measure the test line at a predetermined time, e.g. 20 minutes,
8) Insert the cassette, control line end first, into the holder and place the smartphone on the holder,
9) Press the Expand Tab on the App and manually align the test line with the circle in the center of the image,
10) the App will calculate the level of infection and display the results after the predetermined time,
11) Read the infection level as undetected (Ct>35), infected not contagious (Ct=26-34), contagious (Ct=20-25), or highly contagious (Ct<20),
12) Send descriptive results to friends, family, employers, co-workers, and or physician as needed or desired.

The results could also be automatically sent to city, state and national health agencies with Ct values.

The following is a continuation of Example Two that represents an alternative analysis. For example, more accurate Ct values may be obtained using a measurement of the control line as well as the test line. Specifically, the measured reflectance of the test line is divided by the sum of the reflectances of the test line plus the control line. In this case, Steps 8 and 9 above becomes two steps, viz. the cassette is inserted control line first to measure the test line, and vice versa to measure the control line. An alignment plate on the bottom of the cassette (FIG. 2, item 25) will ensure alignment of the lines below the camera for each measurement. The procedure above continues with Step 10.

The following is another continuation of Example Two that represents alternative sampling using nasal mucus. It is of course realized that the SARS-CoV-2 virus continues to mutate. For example, the above procedure might work well for one variant, but not for a second variant, e.g. Delta and Omicron, respectively. Virions of the second variant may occur in greater number in the nasal cavity, such that Step 3 above is replaced by collecting a nasopharyngeal sample by swabbing both nasal cavities in a circular motion 10 to 20 times to achieve the desired quantitative accuracy. The procedure above continues with Step 4.

The following is another continuation of Example Two that represents alternative sampling using breath. As described above, future mutations of the SARS-CoV-2 virus may yield higher concentrations of samples in exhaled breath. In this case the conjugate pad with probes on the LFA is replaced by a vial that contains the probes. Referring to the apparatus in FIG. 3, for Step 3 above, the person takes a deep breath and blows into a balloon item 35 within a rigid box item 36 that defines the volume, and turns a valve item 37 to seal a breath sample inside. The valve is then connected to a vial containing probes item 38 and opened until the balloon collapses, and then the valve is closed. The vial is gently shaken and allowed to sit for 5 minutes, and the procedure continues with Step 6 above.

The above analysis of breath may also be used to detect a wide variety of respiratory viruses, and in settings other than at-home. For example, the detection of common hospital acquired pathogens, Methicillin-resistant Staphylococcus aureus (MRSA), M. tuberculosis and Streptococcal pneumonia. In this case, the same basic procedures are performed, except by hospital staff as a point-of-care analysis. The quantitative results could be used to quarantine patients.

Thus, it can be seen that the present invention provides a novel method and apparatus for detecting, identifying, quantifying, and analyzing, in an analyte sample, target analyte(s) that bind to target analyte-specific binding agents. More specifically, it provides such a method and apparatus wherein analyses are effected by measurement of the intensity of a flow assay test line in combination with or without the control line by a smartphone camera and App, with substantial selectivity, sensitivity, and speed.

1. A method for the detection, identification, quantitation, and analysis, using a flow assay strip and a smartphone, of “at least one” designated target analyte, comprising the steps:
providing a signal-enhancing probe composed of a metal, coated with a reporter molecule, encapsulated in a silica outer layer, that is functionalized with “at least one” first binding agent that has a specific capability for binding thereto “at least one” designated target analyte;
providing a flow assay strip comprised of a material supporting, in sequence, a sample pad for sample deposition, a conjugate pad containing the probes, a test line functionalized with a second binding agent that also binds the target analyte, and is of the same type as the first binding agent, and may be the same, a control line functionalized with a third, but opposite type of binding agent, to bind excess probes, and a wicking pad; all of which have sufficient porosity to promote flow by capillary action from the sample pad to the wicking pad;
obtaining an analyte sample possibly containing said “at least one” designated target analyte;
adding said analyte sample to the flow assay strip sample pad;
establishing or maintaining conditions sufficient to effect sample flow and binding of said “at least one” designated target analyte to said “at least one” first binding agent of said one functionalized probe at the conjugate pad;
establishing or maintaining conditions effective to cause the target analyte, now bound to a probe, to flow and bind to a second binding agent at a test line forming a sandwich structure;
establishing or maintaining conditions effective to cause excess functionalized probes to flow and bind to a third binding agent at the control line;
providing a smartphone containing a camera capable of (a) displaying and recording a real-time image of the flow assay strip, (b) accessing the Cloud to download an App and download and upload data;
providing a smartphone App to measure the intensity obtained from, but not limited to the real-time and recorded image of one of the following: (a) the test line, and (b) the test line and the control line;
quantifying said target analyte by comparing the smartphone measured intensity obtained from, but not limited to the image of one of the following: (a) the test line, and (b) the test line and the control line to a corresponding concentration calibration curve contained in the App;
said corresponding concentration calibration curve predeveloped from smartphone measurement of intensities of a series of analyte samples of known concentration encompassing the quantitative range of the target analyte, based on, but not limited to one of the following: (a) the intensity of the test line, (b) the intensity of the test line divided by the intensity of the control line, and (c) the intensity of the test line divided by the intensity of the test line plus the intensity of the control line;
wherein, the intensity is one of, but not limited to the following measured at the test line and the control line: (a) the absorbance, (b) the absorption, (c) the optical density, (d) reflectance, (e) the reflection, and (f) the red, green, and blue color values individually and in any combination;

2. The method of claim 1, wherein analysis is obtained by using the test line and the control line as follows: a measurable intensity at both lines indicates positive detection and identification of the target analyte, a measurable intensity at only the control line indicates negative detection of the target analyte, and no measurable intensity at the control line indicates a failed measurement.

3. The method of claim 1, wherein said calibration curve to be used to quantify a target analyte, is downloaded to the smartphone by scanning a barcode or a QR code on the sample package using the aforementioned App on the smartphone.

4. The method of claim 1, wherein said App quantifies the measured intensities of the target analyte in specific units, including, but not limited to ng/mL, ng/cc, mg/L, part per billion (ppb), part per million (ppm), colony forming units (cfu), cycle-to-threshold (Ct) values, and in general terms, including, but not limited to low, high, good, bad, not-infected, infected, not contagious, contagious, and the like.

5. The method of claim 4, wherein said analysis is displayed on the smartphone screen in an easy to understand format, and can be exported using the smartphone in the following forms, including, but not limited to a phone call, text message, and email message.

6. The method of claim 5, wherein said analysis can be exported to one or more selected persons and one or more selected databases directly or indirectly, such as a Cloud database, manually or automatically, including, but not limited to: (a) sending a treatment drug concentration to the person's physician, their medical provider's database, and drug study databases, and (b) the Ct value for a virus, such as SARS-CoV-2, to a person's family, employer, and co-workers, and data bases of local, town, city, district, state, regional, and national health agencies.

7. The method of claim 1 wherein said flow assay strip is selected from the group consisting of (a) a lateral flow assay strip and (b) a vertical flow assay strip, preferably a lateral flow assay strip most often incorporated into a plastic or cardboard cassette.

8. The method of claim 1, wherein a chase buffer is added after the sample to remove unbound probes from the test line in order to improve the measurement, wherein the chase buffer is selected from the group of water, pH buffers, phosphate buffered saline, NaCl, EDTA, borate, Tween 20, poly(vinyl) alcohol, poly(vinyl) pyrrolidone, surfactants BSA, casein, and other chemicals and biologicals used by those skilled in the art.

9. The method of claim 1, wherein said flow assay strip does not have a conjugate pad containing said probes, and wherein the probes are added to the analyte sample, allowed to bind, then added to the flow assay strip by a means selected from the group of deposition on the sample pad, and placing the sample pad end of the flow assay strip into a sample.

10. The method of claim 1, wherein said probe metal is selected from the group consisting of copper, gold, silver, nickel, platinum, rhodium, iron, ruthenium, cobalt, nickel, palladium, and alloys and mixtures thereof, preferably gold or silver.

11. The method of claim 1, wherein said probe metal is selected from the group of particulate, colloidal, star, and urchin form having submicron dimensions.

12. The method of claim 1, wherein said reporter molecule is a dye or a thiol group containing molecule.

13. The method of claim 1, where said flow assay strip is a substantially planar strip fabricated with a composition selected from the group of nitrocellulose, paper, plastic, a combination of these materials, or any suitable material with sufficient porosity to allow flow.

14. The method of claim 1, wherein said “at least one” first binding agent is attached to said probe and by means of covalent, ionic, or hydrogen bonding, or by van der Waals or electrostatic interactions between charged, polar, hydrophobic, or hydrophilic chemical groups on the surface of said “at least one” binding agent.

15. The method of claim 1, where said “at least one” designated target analyte is attached to said “at least one” first and second binding agent by a means selected from the group consisting of covalent, ionic, and hydrogen bonding, by van der Waals and electrostatic interactions between charged, polar, hydrophobic, and hydrophilic chemical groups on the surface of said “at least one” binding agents.

16. The method of claim 1, wherein said “at least one” second and third binding agents are attached to said flow assay strip by means of covalent, ionic, or hydrogen bonding, or by van der Waals or electrostatic interactions between charged, polar, hydrophobic, or hydrophilic chemical groups on the surface of said “at least one” second and third agents.

17. The method of claim 1, wherein said “at least one” first binding agent is attached to said probe, and said “at least one” second and third binding agents are attached to the flow assay strip by means of covalent, ionic, or hydrogen bonding, or by van der Waals or electrostatic interactions between charged, polar, hydrophobic, or hydrophilic chemical groups on the surface of said three binding agents.

18. The method of claim 1, wherein a linker, selected from the group of a chemical, biochemical and biological, is interposed for attaching said “at least one” first binding agent to said probe, and said “at least one” second and third binding agents to said flow assay strip.

18. The method of claim 17, wherein a linker chemical is selected from the group of cysteine, a thiol group of a chemical or biochemical, 4-(N-Maleimidomethyl)cyclohexane-1-carboxylic acid 3-sulfo-N-hydroxysuccinimide ester sodium salt, N-5-azido-2-nitrobenzoyloxysuccinimide, 3-aminopropyl trimethoxy silane, NHS, 1-ethyl-3-(3-dimethylaminopropyl) carbodiimide hydrochloride, and N-hydroxysulfosuccinimide.

20. The method of claim 1, wherein said analyte sample is obtained from, but not limited to one of the following sources: air, water, soil, humans, animals, food, feed and surfaces.

21. The method of claim 20, wherein the human analyte sample is selected from the group of whole blood, blood plasma, blood serum, exhaled breath condensate, breast milk, nasal mucus, nasopharyngeal mucus, saliva, throat sputum, semen, spinal fluid, sweat, tear drops, urine, or other biological matter.

22. The method of claim 1, wherein each of said “at least one” designated target analyte is a chemical, biochemical, or biological substances.

23. The method of claim 22, wherein each of said “at least one” designated target analyte is a chemical substance selected from the group consisting of any general chemical, drug, explosive, radionuclide, pesticide, inorganic or organic pollutant, and their associated precursors or break-down products (e.g. hydrolysis products, metabolites, etc.).

24. The method of claim 23, wherein drugs include, but are not limited to, antiepileptic, antiarrhythmic, antibiotic, antidepressants, antidiabetics, bronchodilator, chemotherapy, immunosuppressant, HIV and substance abuse treatment drugs.

25. The method of claim 24, wherein treatment drugs include, but are not limited to carbamazepine, phenobarbital, phenytoin, valproic acid, digitoxin, digoxin, lidocaine, nacetylprocainamide, procainamide, gentamicin, tobramycin, vancomycin, lithium, theophylline, cyclosporine, mycophenolic acid, azacitidine, 5-fluorouracil, 6-mercaptopurine, capecitabine, cladribine, chlorfarabine, cytarabine, decitabibe, sirolimus, tacrolimus, buprenorphine, methadone, naloxone and naltrexone.

26. The method of claim 1, wherein each of said “at least one” designated target analyte and said “at least one” first, second and third binding agents are biological substances selected from the group consisting of amino acids, nucleic acids, nucleotides, nucleosides, oligonucleotides, peptides, proteins, lipids, polysaccharides, haptens, antibodies, antigens, affirmer proteins, bacteriophages, biomarkers, enzymes, steroids, hormones, lectins, aptamers, and fragments and polymers thereof.

27. The method of claim 1, wherein said “at least one” first and second binding agents and said “at least one” designated target analyte are sometimes paired with one another for effective interbonding, such pairs are selected from the group consisting of (a) antibodies and antigens, (b) peptides and biologicals, (c) drug receptors and drugs, (d) enzymes and their specific biochemical substrates, (e) carbohydrates and lectins, and (f) nucleic acid sequences and their complements.

28. The method of claim 1, wherein said “at least one” target analyte is a disease-causing pathogen.

29. The method of claim 28, wherein said disease-causing pathogen is selected from the group consisting of Acute Flaccid Myelitis (AFM), Burkholderia mallei, coronaviruses, including Severe Acute Respiratory Syndrome (SARS)-229E, -CoV-2, -HKU1, -OC43, and -NL63, Corynebacterium diphtheriae, Enteric viruses, Enterobacter aerogenes, Equine encephalitis, hemorrhagic fevers, Hepatitus A through E, herpes simplex viruses 1 and 2, human immunodeficiency virus, influenza, Legionella, Lyme borreliosis, measles, meningitis, mumps, methicillin-resistant Staphylococcus aureus (MRSA), Middle East Respiratory Syndrome (MERS), Mycobacterium tuberculosis, Mycoplasma pneumonia, Multisystem Inflammatory Syndrome (MIS), Neisseria gonorrhoeae, Neisseria meningitidis, Norwalk virus, members of the Orthomyxoviridae family, pertussis, Pneumococcal Disease, rabies virus, Respiratory Syncytial Virus Infection (RSV), rhinoviruses, Rubella virus, saxitoxin, sepsis, Shigella subspecies, dysenteriae, Staphylococcus aureus, Staphylococcal and Streptococcus pneumonia, Swine disease, Treponema pallidum, Vibrio cholerae, Varicella zoster virus, West Nile virus, and Yellow fever.

30. The method of claim 1, wherein said “at least one” target analyte is a foodborne or waterborne pathogen.

31. The method of claim 30, wherein said foodborne or waterborne pathogen is selected from the group consisting of Aeromonas hydrophilia, Bacillus cereus, Campylobacter jejuni, Clostridium botulinum B and C, Clostridium difficile and perfringens, Cryptosporidium parvum, Escherichia coli, Giardia lamblia, Hepatitis A, Listeria monocytogenes and spp., Salmonella enterica, typhimurium and spp., Shigella, and Yersinia enterocolitica.

32. The method of claim 1, wherein said “at least one” target analyte sample is a biological agent.

33. The method of claim 32, wherein said biological agent is selected from the group consisting of Bacillus anthraces, Clostridium botulinum A, Dengue fever, Ebola virus, Francisella tularensis, Leishmania genus, Marburg virus, Mycobacterium leprae, Plasmodium genus, Puumala hantavirus, Ricin toxin, Variola virus, and Yersinia pestis.

34. The method of claim 1, wherein said “at least one” target analyte is selected from the group consisting of the SARS CoV-2 virion, the spike glycoprotein or the S1 and S2 subunits of the spike protein on the surface of the SARS CoV-2 virion, a membrane protein, the nucleocapsid protein contained inside the envelope of the SARS CoV-2 virion, the RNA and RNA unique sequences of the SARS CoV-2 virus, the antibody created by the human immune system to deactivate the SARS CoV-2 virions, and any subunit and molecular feature unique to the SARS CoV-2 virion.

35. The method of claim 1, wherein said establishing conditions to effect binding of said “at least one” designated target analyte to said “at least one” binding agent of said one functionalized probe includes pretreatment of the analyte sample prior to addition to the flow assay strip.

36. The method of claim 35, wherein in pretreatment includes, but is not limited to, the use of chemicals, biochemicals, filters, heat or light to aid in the separation of the target analyte from the analyte sample.

37. The method of claim 36, wherein in pretreatment chemicals and biologicals are selected from the group consisting of acids, bases, buffers, solvents, digesting agents, lysing agents, coagulants, enzymes, amino and nucleic acids, nucleotides, nucleosides, oligonucleotides, proteins, lipids, polysaccharides, haptens, antibodies, antigens, affirmer proteins, bacteriophages, biomarkers, steroids, hormones, lectins, aptamers, and fragments and polymers thereof.

38. Apparatus, in the form of a kit of components and a smartphone, for use in the detection, identification, quantitation and analysis, of “at least one” designated target analyte in an analyte sample, by a smartphone camera measured intensity of an image of the test line, and in some preferred cases the measured intensities of an image of the test line and the control line of a flow assay strip, comprising:
a packaging means for the containment of a multiplicity of components;
“at least one” sample collection device selected from the group consisting of a non-cotton swab, a spatula, a balloon for exhaled breath, an open-ended tube like a straw, a vial, a vial containing probes, a centrifuge tube, a cup, a lancet for finger needle prick collection of blood, and a syringe with needle;
“at least one” sample holding device selected from the group consisting of a vial, a centrifuge tube, and a cup;
“at least one” sample transfer device selected from the group consisting of an eyedropper, pipette, autopipette, a syringe, a vial, a centrifuge tube, and a cup;
“at least one” flow assay strip consisting of a sample pad, a conjugate pad containing probes, a test line functionalized to bind the target analyte, a control line functionalized to bind excess probes, and a wicking pad;
a holder to position “at least one” test line and, as required, “at least one” test line and control line of “at least one” flow assay strip at the measurement point of a smartphone camera;

39. The apparatus of claim 38, wherein said flow assay strip is selected from the group consisting of (a) a lateral flow assay strip and (b) a vertical flow lateral flow assay strip, preferably a lateral flow assay strip, most often incorporated into a plastic or cardboard cassette.

40. The apparatus of claim 38, wherein said flow assay strip does not have a conjugate pad containing said probes, and wherein the probes are added to the analyte sample, allowed to bind, then added to the flow assay strip by a means selected from the group of (a) deposition on the sample pad and (b) placing the sample pad end of the flow assay strip into the sample.

41. The apparatus of claim 38, wherein said components include “at least one” reagent holding device with a cap selected from the group consisting of a vial, a tube, and a centrifuge tube, and “at least one” reagent in a reagent holding device used to pretreat the analyte sample prior to addition to the flow assay strip.

42. The apparatus of claim 38, wherein said “at least one” reagent used to separate the target analyte from the analyte sample is selected from the group consisting of acids, bases, buffers, solvents, digesting agents, lysing agents, coagulants, enzymes, amino and nucleic acids, nucleotides, nucleosides, oligonucleotides, proteins, lipids, polysaccharides, haptens, antibodies, antigens, affirmer proteins, bacteriophages, biomarkers, steroids, hormones, lectins, aptamers, and fragments and polymers thereof.

43. The apparatus of claim 38, wherein said components include “at least one” reagent holding device with a cap selected from the group consisting of a vial, a tube, and a centrifuge tube, and “at least one” reagent in a reagent holding device is a chase buffer used to remove unbound probes from the test line in order to improve the measurement, selected from the group of water, pH buffers, phosphate buffered saline, NaCl, EDTA, borate, Tween 20, poly(vinyl) alcohol, poly(vinyl) pyrrolidone, surfactants BSA, casein, and other chemicals and biologicals used by those skilled in the art.

44. The apparatus of claim 38, additionally includes “at least one” mixing vial component contained by said packaging means.

45. The apparatus of claim 38, additionally includes a device to separate the target analyte from the analyte sample or from a pretreated sample, selected from the group of a filter, a solid phase extractor, a heater, and a centrifuge.

46. The apparatus of claim 38, additionally may include a flow assay strip holder, selected from the group of a smartphone attachment, a box and a stand, that aligns the test line and, when required, the control line of the flow assay strip in front of the smartphone camera measurement point.

47. The apparatus of claim 38, additionally includes the ability of the smartphone to magnify and focus the test line image and as required the control line image.

48. The apparatus of claim 38, additionally includes an aligning object, selected from the group of a bull's eye, cross hairs, circle, square, rectangle and the like, that is superimposed on the center of the camera view, and used to define the camera measurement point.

49. The apparatus of claim 38, additionally includes a sliding tray to which a cassette holding a FAS is placed and wherein manual placement of the slide in the holder in one direction aligns the test line with the camera measurement point, and placement of the slide in the opposite direction aligns the control line with the camera measurement point.

50. The apparatus of claim 38, wherein a sliding tray to which a cassette holding a FAS is placed automatically moves the slide holding the FAS cassette from the test line to the control line using the smartphone App through communication with the holder.

51. The apparatus of claim 38, wherein said smartphone App scans the camera image of the test line to detect and measure the maximum intensity, and when required the control line, to calculate the target analyte concentration.

52. The apparatus of claim 38, wherein said holder employs a means, selected from a cover and an enclosure, to block ambient light, and automatically turns on the smartphone camera and controls the amount of the light for the measurement of the intensity of the test line, and, when required, the control line.

53. The apparatus of claim 50, includes an automated XY positioning stage that contains a support with slots that hold multiple FASs, and is used to position the test and control lines for each FAS in front of the smartphone camera measurement point.

54. The apparatus of claim 38, wherein the sample flow along the FAS from the sample pad to the wicking pad occurs within a period of 30 minutes, preferably within 20 minutes, and ideally within 10 minutes.

55. The apparatus of claim 38, wherein said total measurement of both the test and control lines combined occurs within 1 minute, preferably within 30 seconds, and ideally, within 10 seconds, performed after the sufficient flow of the sample to the wicking pad.

56. The apparatus of claim 38, wherein said test kit, holder, and smartphone afford ease of use and portability, for at-site measurements, such as at-home, in an ambulance, at the bedside of a hospitalized patient, at local medical clinics, at the site of an infectious outbreak, at laboratories, and at food processing facilities.
